(19) United States
(12) Patent Application Publication
(10) Pub. No.: US 2006/0079897 A1
(43) Pub. Date: Apr. 13, 2006

(54) Apparatus and methods for magnetic alteration of anatomical features

(76) Inventors: Michael R. Harrison, San Francisco, CA (US); Richard J. Fechter, San Rafael, CA (US); Arthur Moran, San Bruno, CA (US)

Correspondence Address: JOHN P. O'BANION;O'BANION & RITCHEY LLP, 400 CAPITOL MALL SUITE 1550, SACRAMENTO, CA, 95814, US

(21) Appl. No.: 11/222,517

(22) Filed: Sep. 7, 2005

Related U.S. Application Data
(63) Continuation-in-part of application No. 10/954,995, filed on Sep. 29, 2004.

(51) Int. Cl. A61F 2/30

(52) U.S. Cl. 606/61

(57) ABSTRACT

Methods and apparatus for incrementally manipulating a body member of a patient are disclosed. The apparatus has a magnetic implant adapted to be received on a location of the body member, a form external to the patient, and a magnetic member coupled to the form, wherein the magnetic member generates a magnetic force between the implant and the form to incrementally manipulate the body member. The implant and external magnetic member are preferably rare earth magnets or an array of rare earth magnets, and are configured to generate an attractive or repulsive force between the implant and the platform to reposition, reorient, deform, or lengthen the body member.

### CROSS-REFERENCE TO RELATED APPLICATIONS

This application is a continuation-in-part of copending application Ser. No. 10/954,995 filed on Sep. 29, 2004, incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C. F. R. § 1.14.

### BACKGROUND OF THE INVENTION

1. Field of the Invention

This invention pertains generally to apparatus and methods for magnetically manipulating body structures and more particularly to performing corrective procedures on a patient via incremental magnetic loading.

2. Description of Related Art

Anatomical deformities occur in the general populous in a number of different forms and from a variety of causes. Examples of skeletal deformities include, pectus excavatum, scoliosis, club feet, and numerous forms of skeletal dysplasia. These conditions are treated in a variety of different manners from braces to surgery, with sometimes minimal efficacy.

The defect known as pectus excavatum, or funnel chest, is a congenital anomaly of the anterior chest wall. The excavatum defect is characterized by a deep depression of the sternum, usually involving the lower half or two thirds of the sternum, with the most recessed or deepest area at the junction of the chest and the abdomen. The lower 4-6 costal or rib cartilages dip backward abnormally to increase the deformity or depression and push the sternum posterior or backward toward the spine. Also, in many of these deformities, the sternum is asymmetric or it courses to the right or left in this depression. In many instances, the depression is on the right side.

Pectus excavatum with significant deformity occurs in approximately 1 out of every 2000 births. The deformity may be present at birth but is often noted after several years of age and usually worsens during rapid growth around puberty. Because of the pressure of the sternum and cartilages, defect also pushes the midline structures so that the lungs are compressed from side to side and the heart (right ventricle) is compressed. Severe lesions have a major effect on thoracic volume and pulmonary function but the principal motivation for repair is the deformity itself. It does occur in families and thus, is inherited in many instances. Other problems, especially in the muscle and skeletal system, also may accompany this defect. In approximately ⅕ of the patients, scoliosis is present. The regression or any improvement in this defect rarely occurs because of the fixation of the cartilages and the ligaments. When one takes a deep breath or inspires, the defect is usually accentuated.

Pectus excavatum can be repaired surgically using an open approach in which the malformed costal cartilages are resected and the sternum forcibly held in place with a metal strut. In another approach, described in U.S. Pat. No. 6,024,759, the sternum is forced into a corrected position often under great tension, and held in place with a metal strut. Both can achieve good results but at the cost of considerable morbidity: an operation under general anesthesia followed by a 4-7 day hospital stay required for pain control usually by continuous epidural analgesia. Several more weeks of moderate to severe discomfort are typical and complications from the sternum held forcibly against the metal strut are not infrequent. It is necessary to leave the bar in place for a year or more before it is removed in another procedure. Total cost usually reimbursed by third party payers averages more than $30,000.

The problem with all currently available pectus excavatum surgical repairs is that they attempt to achieve immediate total correction and fixation often under considerable tension. A better approach would be the gradual step-by-step correction of the deformity by applying a smaller force over a longer period of time.

Another skeletal deformity, scoliosis, is a condition in which an individual has an abnormal spine curvature. Generally, some curvature in the neck, upper trunk and lower trunk is normal. However, when there are abnormal side-to-side (lateral) curves in the spinal column, the patient is generally diagnosed as having as scoliosis.

Orthopaedic braces are typically used to prevent further spinal deformity in children with curve magnitudes within the range of 25 to 40 degrees. If these children already have curvatures of these magnitudes and still have a substantial amount of skeletal growth left, then bracing is a viable option. The intent of bracing, however, is to prevent further deformity, and is generally not used to correct the existing curvature or to make the curve disappear.

Surgery is an option used primarily for severe scoliosis (curves greater than 45 degrees) or for curves that do not respond to bracing. The two primary goals for surgery are to stop a curve from progressing during adult life and to diminish spinal deformity.

Although there are different techniques and methods used today for scoliosis surgery, all of them involve fairly invasive procedures with considerable patient morbidity. One frequently performed surgery involves posterior spinal fusion with instrumentation and bone grafting, which is performed through the patient's back. During this surgery, the surgeon attaches a metal rod to each side of the patient's spine by anchor's attached to the vertebral bodies. The spine is then fused with a bone graft. The operation usually takes several hours and the patient is typically hospitalized for a week or more. Most patients are not able to return to school or for several weeks after the surgery and cannot perform some pre-operative activities for up to four to six months.

Another surgery option for scoliosis is an anterior approach, wherein the surgery is conducted through the chest walls instead of entering through the patient's back. During this procedure, the surgeon makes incisions in the patient's side, deflates the lung and removes a rib in order to reach the spine. The anterior spinal approach generally has quicker patient rehabilitation, but usually requires bracing for several months after this surgery.

Infants born with esophageal atresia (EA) have a portion missing (a gap) in their esophagus, the tube that goes from the back of the mouth to the stomach. With part of the esophagus missing, the baby cannot swallow food or even its own saliva. These defects are discovered either before birth by ultrasound examination or very shortly after birth in the delivery room.

EA takes many forms and there is a lot of variation in this group of defects. The spectrum of the defects including the five main types, are shown in FIG. 27A-E. Many additional variations, however, can occur.

FIGS. 27 A-E illustrate a diagram of the main types of EA. FIG. 27A shows an example of type A EA, which is often called pure EA. With a type A condition, the esophagus 550 has a gap or separation between the upper 552 and lower portions forming atresias, blind upper and lower esophageal pouches, next to the ringed trachea 556 and the bronchi 558 which lead to each lung.

Type B EA is characterized by a connection (fistula 560) between the upper pouch 552 and the trachea 556, known as a tracheoesophageal fistula (TEF). Type C is by far the most common form of EA and has a fistula 562 between the lower esophagus 554 and the trachea 556 with a blind upper pouch 552. A rare form is type D with two TEFs, one 560 between the upper esophageal segment 552 and the trachea 556, and the other 562 between the lower esophageal segment 554 and the trachea 556. Type E has only a TEF and no EA. The E-fistulas are divided surgically and nothing further needs to be done to the esophagus which is intact and reaches normally to the stomach.

For all the babies with EA, with or without a TEF, the missing segment between the two ends of the esophagus 550 must be connected for normal eating. Most of these babies with EA will have an upper esophageal pouch 552 that ends blindly and a lower esophageal segment 554 that connects into the trachea 556 at fistula 562 (FIG. 27C). For these cases the operation is straight forward and usually completed satisfactorily. The basic operation consists of joining (sewing) together the two ends of the esophagus (an anastomosis) using fine sutures. If a TEF is present, the fistula 562 is detached and the hole in to the trachea 556 repaired. The two ends of the esophagus 554, 552 are opened and fine sutures used to bring the two ends together. When there is a moderate gap between the two esophageal ends, these sutures are used to pull the ends together.

For 8-10%, however, there will not be a connection between esophagus and trachea (no TEF, as shown in FIG. 27A). For these infants, the distance between the two esophageal ends 552, 554 is usually longer (a long gap EA), and the operation may be much more difficult and the results not always good. Similarly, when the TEF is only between the upper pouch 552 and the trachea, the lower end 554 of the esophagus tends to be short leaving a long gap between the ends (FIG. 27B).

For long gap EA, the current approach is suture the ends of the esophagus for several days of either internal or external traction. This method requires the child to be on a ventilator and heavily sedated so they don't tear traction sutures loose, as well as repeated re-opening of the wound to re-apply pressure once the esophageal members have lengthened. Other procedures include an esophageal substitute such as stomach or colon.

Another procedure involving physical reformation of a body structure that has dramatically increased in popularity over the years is cosmetic, or plastic surgery. Most cosmetic surgery procedures are highly invasive and are associated with numerous risks, complications and failed results.

For example, rhinoplasty, or surgery to reshape the nose, is one of the most common of all plastic surgery procedures. During a typical rhinoplasty surgery, the skin of the nose is separated from its supporting framework of bone and cartilage, which is then sculpted to the desired shape. The nature of the sculpting will depend on the problem and the surgeon's preferred technique. Finally, the skin is redraped over the new framework.

When the surgery is complete, a splint will be applied to help your nose maintain its new shape. Nasal packs or soft plastic splints also may be placed in your nostrils to stabilize the septum, the dividing wall between the air passages.

Post surgery complications often include swelling, aching and headaches, often requiring pain medication prescribed by your surgeon. At least a day bed rest with head elevated is often required. Generally, the patient will not be able to return to work for at least 2 weeks. Many procedures have risks including injury to the nerves that control facial muscles or feeling, infection, bleeding, poor healing, excessive scarring, and asymmetry or change in hairline.

Other cosmetic surgery procedures, such as mentoplasty (chin surgery to reshape the chin either by enhancement with an implant or reduction surgery on the bone), ear surgery, (otoplasty), face lifts and browlifts are similarly invasive with many of the same complications and risks.

Several procedures will also involve implants on the chin, cheeks and jaw. Complications associated with implants include shifting or imprecise positioning of implant, or infection around it, requiring a second operation or removal. In addition, excess tightening and hardening of scar tissue around an artificial implant (“capsular contracture”) may occur, causing an unnatural shape.

A final form of external body manipulation, body piercing, which is most commonly done in the ear-lobe for earrings, also has drawbacks. Many piercings leave the subject with a wound open to infection, closure or other complications. Magnetic earrings that are currently available, e.g. earrings held to the ear via magnets on opposing sides of the earlobes, attempt to solve many of these problems. However, since the magnetic force is outside the body, the attractive force holding the earring in place is weak and/or discomforting.

For these reasons it would be desirable to provide improved apparatus and methods for repositioning bone structures, by applying a corrective force to the bone structure, which could be gradually adjusted much like orthodontic tooth braces.

It would be further desirable to provide a device that applies a corrective force to reposition a body member without a mechanical force that requires piercing of the skin, thereby limiting the specter of infection and wound problems.

In addition, it would be desirable to provide a device for repositioning bones structures having tension-sensing technology to allow measurement of the force applied to correct all types of asymmetric deformities and allow protection of skin against pressure damage.

It would further be desirable to provide improved devices and methods for minimally invasively treating pectus excavatum.

In addition, it would be desirable to provide improved devices and methods for minimally invasively treating scoliosis.

In addition, a further object is to provide an improved device and method for treating esophageal atresia.

Yet a further object is an improved device for performing cosmetic surgery for various locations in the patient's body.

At least some of these objectives will be met with the inventions described hereinafter.

### BRIEF SUMMARY OF THE INVENTION

The present invention comprises apparatus and methods for altering the position, orientation, growth or development of body parts and organs by magnetic forces to apply a steady sustained force over time. The invention uses magnetic force fields that may be used to correct a number of anatomic deformities, including, but not limited to: pectum excavetum, pectus carinatum, scoliosis, club feet, cranial/facial anomalies or defects, skeletal dysplasias, cartilaginous deformities/dysphasia, and joint deformities/dysphasia. The invention may also be used to incrementally lengthen bone or apply bone compression to promote healing.

An aspect of the invention is an apparatus for incrementally manipulating an internal body member of a patient. In one embodiment, the apparatus comprises magnetic implant adapted to be received on a location of the body member, a platform external to the patient, and a magnetic member coupled to the platform, wherein the magnetic member generates a magnetic force between the implant and the platform to incrementally manipulate the body member. The implant and external magnetic member preferably comprise a rare earth magnet or array of rare earth magnets, and are configured to generate an attractive or repulsive force between the implant and the platform to reposition, reorient, deform, or lengthen the body member.

In one aspect of the invention, the implant is adapted to be received on a location of the sternum to treat pectus excavatum. In this configuration the platform comprises a chest plate adapted to be positioned exterior to the patient's chest. The magnetic member is coupled to the chest plate to generate an attractive force between the implant and the chest plate to incrementally reposition the sternum.

The implant is preferably adapted to be received on a posterior surface on the sternum. The implant generally comprises an internal magnet and a casing to enclose the internal magnet. The casing may be made from any rigid biocompatible material capable of withstanding the forces of the magnet without significant deformation, such as high-grade medical epoxy or similar material used in the art.

In a preferred embodiment, the implant is attached to the sternum using a plurality of sutures, wherein the sutures are looped through a plurality of holes in the implant casing and around the sternum to attach the implant to the posterior surface of the sternum.

In one embodiment, the platform chest plate generally has a concave inner surface to allow the sternum to deform outwardly from the chest. The platform may also have an adjustable stage coupled to the chest plate, wherein the magnetic member is mounted on the stage. A plurality of adjustment members may be coupled to the stage to adjust the orientation and position of the magnetic member with the implant.

In another embodiment of the invention, a plurality of sensors and a strain gauge may be coupled to the chest plate, with the strain gauge measuring the force applied to one or more locations on the platform.

In most cases the attractive force of the magnets support the chest plate to the patient's chest. However, a chest strap may also be used to support the chest plate to the patient's chest.

In another aspect of the invention, the implant is adapted to be received on a location of a vertebrae of the patient's spine to treat scoliosis or other spinal disorders. In this configuration, the platform comprises a support adapted to be positioned exterior to the patient's torso. Generally, the magnetic member is coupled to the support such that the magnetic member generates a magnetic force between the implant and the plate to incrementally reposition the spine. The magnetic member and the implant may be configured to generate an attractive or repulsive force between the implant and the magnetic member.

Where the patient has an abnormal curvature of the spine, the implant is preferably configured to be received on a vertebrae located at an apex of the abnormal curvature. The support may be positioned such that the magnetic force incrementally repositions the spine to remove the abnormal curvature. The implant and the magnetic member may also be configured to impart a torsional force on the vertebrae to incrementally reorient the spine.

In one embodiment, a bone screw is threaded into the vertebrae to rigidly couple the implant to the vertebrae.

According to another aspect of the invention, a method for incrementally repositioning an internal body member of a patient comprises installing a magnetically responsive implant to a location on the internal body member; positioning a platform exterior the patient to generate a magnetic field, the magnetic field effecting an magnetic force between the implant and the platform, and manipulating the body member to a first state as a result of the generated magnetic force.

In a preferred embodiment, the method also includes adjusting the magnetic field to one or more intermediate settings, manipulating the body member to one or more intermediate state as a result of the attractive force generated by the one or more adjusted magnetic field settings, adjusting the magnetic field to a final setting; and manipulating the body member to a final state as a result of the attractive force generated by the final magnetic field setting.

The step of generating a magnetic field may comprise generating an attractive or repulsive force between the implant and the platform. The body member may be manipulated by repositioning the body member to a first position, deforming the body member to a first shape, or lengthening the body member to a first length. Repositioning the sternum may comprise deforming one or more cartilages connected to the sternum as a result of the attractive force, or deforming the shape of the sternum as a result of the attractive force.

In one aspect of the invention, manipulating a body member comprises manipulating the patient's sternum. In such a configuration, installing a magnetically responsive implant comprises attaching an internal magnet to a posterior location on the sternum. Positioning a platform is achieved by manipulating a stage housing an external magnet, the stage being coupled to a chest plate. A plurality of adjustment members may be used to adjust the position and orientation of the external magnet with respect to the internal magnet, thereby effecting the magnitude and direction of the magnetic force between the platform and the implant.

In one aspect of the invention, manipulating a body member comprises manipulating a vertebrae of the patient. In such a configuration, installing a magnetically responsive implant comprises attaching an internal magnet to a location on the vertebrae. The vertebrae may be manipulated by adjusting the magnetic field between the implant and the platform to incrementally reposition the spine. The magnetic field may be adjusted to generate an attractive or repulsive force between the implant and the platform to incrementally reposition the spine. Where the spine has an abnormal curvature, the implant is installed on a vertebrae located at an apex of the abnormal curvature. In such a configuration the vertebrae may be manipulated to incrementally reposition the spine to remove the abnormal curvature. A torsional force may also be imparted on the vertebrae to incrementally reorient the spine.

In a preferred embodiment, installing the implant comprises boring a hole in a pedicle of the vertebrae, and threading a pedicle screw into the pedicle, the pedicle screw configured to rigidly couple the implant to the vertebrae.

In another aspect of the invention method is disclosed for performing a pectus excavatum procedure on a patient having a deformed sternum. The method comprises attaching a magnetically responsive implant to a location on the sternum, and positioning a chest plate exterior the patient's chest to generate a magnetic field, wherein the magnetic field effects an attractive force between the implant and the chest plate. The implant generally comprises a first magnet housed in a biocompatible casing.

The first magnet may be attached to a posterior surface on the sternum by incising a section of the patient's skin over the patient's sternum, separating the xiphoid process from the sternum, dissecting under the sternum and securing the first magnet to the posterior surface of the sternum. One method for securing the first magnet to the sternum comprises drilling a plurality of holes from an anterior location on the sternum to a posterior location on the sternum, and looping a plurality of sutures through the holes in the sternum and through a plurality of holes in the casing housing the first magnet.

According to yet another aspect of the invention, a method for incrementally repositioning a patient's sternum is disclosed. The method comprises installing a magnetically responsive implant to a location on the sternum, positioning a chest plate exterior the patient's chest to generate a magnetic field, wherein the magnetic field effects an attractive force between the implant and the chest plate, repositioning the patient's sternum to a first position as a result of the generated magnetic force, manipulating the magnetic field to one or more intermediate settings, repositioning the patient's sternum to one or more intermediate positions as a result of the attractive force generated by the one or more manipulated magnetic field settings, manipulating the magnetic field to a final setting, and repositioning the patient's sternum to a final position as a result of the attractive force generated by the final magnetic field setting.

According to a further aspect of the invention, an apparatus for incrementally manipulating an internal body member of a patient comprises a magnetically responsive implant adapted to be received on a location of the body member, the implant responsive to a magnetic field, and means for generating an attractive force between the implant and a platform external to the patient to manipulate the body member. The device may further comprise means for adjusting the magnitude and direction of the magnetic force applied between the platform and the implant. The device also has means for securing the implant to a location on the body member.

In a preferred embodiment, the apparatus has a means for detecting the force applied to the platform at a plurality of locations on the platform, such as a strain gauge. The strain gauge may also configured to measure the force at a plurality of locations on the platform.

According to yet another aspect of the invention, a method for incrementally repositioning an internal body member of a patient comprises installing a magnetically responsive implant to a location on the internal body member, positioning a platform exterior the patient to generate a magnetic field, the magnetic field effecting a magnetic force between the implant and the platform, measuring the magnetic force between the implant and the platform; adjusting the platform to tune the magnetic force applied between the implant and the platform; and manipulating the body member to a first state as a result of the generated magnetic force.

In an alternative embodiment of the present invention, an apparatus for manipulating one or more internal body members is disclosed. The apparatus comprises a first elongate member having a driving end and a receiving end, wherein the receiving end of the first member having a recess extending toward the driving end. The apparatus also has a second elongate member having a driving end and a receiving end, the receiving end of the second member having a recess extending toward the driving end. The second member is sized such that the receiving end of the second member is slideably received within the receiving end of the first member. The apparatus further comprises a first magnet coupled to the first member, and a second magnet coupled to the second member, wherein the first and second magnets are configured to repel each other such that an outward magnetic force is generated to the driving ends of the first and second members. The first and second magnets may be positioned within the recesses to change the magnitude of the force generated between the first and second magnets. The first and second magnets may also be configured such that rotation of the first magnet with respect to the second magnet changes the magnitude of the force generated between the first and second magnets.

According to another aspect of the invention, an apparatus for intermittently delivering a force to a body member to incrementally manipulate the body member comprises an implant adapted to be received on a location of the body member, the implant responsive to a magnetic field, a platform external to the patient, and a magnetic member coupled to the platform, wherein the magnetic member generates a magnetic force between the implant and the platform to incrementally manipulate the body member, the magnetic member and the implant configured such that rotation of the magnetic member varies the magnetic force between the implant and the platform.

Yet another aspect is an apparatus for incrementally manipulating an internal lumen of a patient. The apparatus has a first magnetic member configured to be received at a first location on the lumen, a first catheter adapted to deliver the first magnetic member to the first location, and a second magnetic member adapted to be received at a second location of the lumen. The first and second magnetic members are configured to generate a magnetic force to incrementally lengthen at least a portion of the lumen.

In one embodiment, the first and second magnetic members are configured to bring two segments of the lumen together, and may be configured to vary the force applied on the lumen, e.g. by varying its position with respect to the second magnetic member. The first magnetic member may be encased in a biocompatible container such that the first magnet member may be positioned at a plurality of locations inside the biocompatible container. Alternatively, the first magnetic member is responsive to an electric current such that the current may be varied to vary the force applied on the lumen. In either case, the first and second magnetic members may be configured to cycle the force applied on the lumen.

In another embodiment, a pressure sensor is coupled to one of the first and second magnetic members, wherein the force applied to the lumen is adjusted according to a reading from the pressure sensor.

In another embodiment, the apparatus is may be used to treat patients with esophageal atresia, wherein the first and second magnetic members are configured to generate an attractive force to close the gap between the lower esophageal atresia and the upper esophageal atresia. The first and second magnetic members may also be configured to connect the lower esophageal atresia to the upper esophageal atresia, and even anastomose portions of the lower esophageal atresia and the upper esophageal atresia.

In one variation of the current embodiment, the first magnetic member is configured to be inserted trans-orally into the upper esophageal atresia via the first catheter, and the second magnetic member is configured to be inserted into the lower esophageal atresia through the patient's stomach via the second catheter.

Another aspect is a method for incrementally manipulating an internal lumen of a patient, comprising positioning a first magnetic member at a first location on the lumen, positioning a second magnetic member at a second location of the lumen, and generating a magnetic force between the first and second magnetic members to incrementally lengthen at least a portion of the lumen.

In one embodiment, the force applied on the lumen may be adjusted by varying the position of the first magnetic member with respect to the second magnetic member to vary the force applied. Alternatively, the first magnetic member may be responsive to an electric current; such that the current may be adjusted to vary the force applied on the lumen.

To treat esophageal atresia, the first magnetic member may be inserted via a first catheter into an upper esophageal atresia of the patient, and the second magnetic member may be inserted via a second catheter into a lower esophageal atresia of a patient such that an attractive force is generated to close the gap between the lower esophageal atresia and the upper esophageal atresia.

In yet another aspect, an apparatus is disclosed for incrementally manipulating a body member (e.g. nose, ear, cheek, eyebrow, etc.) of a patient. The apparatus includes a magnetically responsive implant adapted to be received on a location of the body member, a form external to the patient, and a magnetic member coupled to the form, wherein the magnetic member is configured to generate a magnetic force between the implant and the form to incrementally manipulate the body member.

In some embodiments, the implant and the magnetic member may be configured to generate an attractive force ore repulsive between the implant and the form to either reposition or deform the body member.

In another embodiment, the implant is adapted to be received at a location in the nose. The form may have a pre-determined shape adapted to be positioned exterior to the patient's nose to incrementally reform the shape of the nose (e.g. be reforming the cartilaginous or bone structure of the nose) to the pre-determined shape.

A further aspect is a method for incrementally manipulating a body member (e.g. nose, ear, cheek, eyebrow, etc.) of a patient, comprising attaching a magnetically responsive implant to an internal location on the body member, the implant responsive to a magnetic field, and positioning a form exterior to the body member to generate a magnetic field such that a corrective force is affected between the implant and the form. The corrective force incrementally adjusts the position of the body member by either deforming one or more cartilages in the body member, or adjusting the shape of the body member.

In some embodiments, the orientation of the external magnet with respect to the implant may be adjusted to vary the direction and/or magnitude of the corrective force between the implant and the form. The corrective force may be an attractive or repulsive force.

In yet another embodiment, the implant may be attached to an internal location on the body member by injecting a solution subcutaneously to the location. The solution may comprise a plurality of medical particles and a bio-compatible liquid gel configured to form into a solid shape over a period of time. A forming magnet is positioned near the location to concentrate the plurality of magnetic particles at the location. Finally, the bio-compatible liquid gel is solidified to fix the location of the plurality of magnetic particles.

A further aspect is a magnetic clasp for attaching an ornamental object to a body member. The clasp has a housing configured to be positioned against a skin surface at a location on the body member adjacent to a magnetically responsive implant positioned under the skin surface of the body member. A magnetic member is coupled to the housing to generate an attractive force between the implant and the housing to retain the housing at the location. The housing may comprises the ornamental object, e.g. an earring, or have means for retaining the ornamental object near the location. Alternatively, the housing is configured to detachably secure the ornamental object near the location.

In another aspect of the invention, a method is disclosed for magnetically attaching an ornamental object to a body member. The method comprises the steps of delivering a magnetically responsive implant to a location under a skin surface of the body member, and positioning a housing against the skin surface at the implant location. The housing has a magnetic member that generates an attractive force between the implant and the housing to retain the housing at the location. Delivery of the magnetically responsive implant may be done by injecting a magnetically responsive solution to the location, and concentrating the magnetically responsive solution at the location by positioning an external magnet near the location. The magnetically responsive solution may be formed into a solid via a bonding agent in the solution.

Yet a further aspect is a method for delivering a magnetically responsive implant into a location of the body by injecting a solution having a plurality of medical particles and a bio-compatible liquid gel subcutaneously to the location, wherein the bio-compatible liquid gel is configured to form into a solid shape over a period of time. A forming magnet is positioned near the location to concentrate the plurality of magnetic particles at the location until the bio-compatible liquid gel is solidified to fix the location of the plurality of magnetic particles. The magnetic particles may also be oriented to align the polarity of the magnetic particles.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only, and where like reference numbers denote like elements:

FIG. 1 is a schematic view of a human sternum with an implant according to the present invention installed under the sternum.

FIG. 2 is a cross-sectional schematic view of the platform of the present invention installed over a patient's chest and an implant installed under the sternum.

FIG. 3 shows an embodiment of the implant of the present invention.

FIG. 4 is a side view of the implant of FIG. 3.

FIG. 5. is a schematic view of a sternum.

FIG. 6. is a cross-sectional view of a sternum with the xiphoid separated from the sternum body.

FIG. 7 is an implant drill guide according to the present invention.

FIG. 8 shows the drill guide of FIG. 7 installed over the sternum.

FIG. 9 illustrates a preferred method for installing a portion of the implant to the posterior surface of the sternum.

FIG. 10 illustrates a portion of the drill guide of FIG. 7 positioned over a second location on the sternum.

FIG. 11 illustrates a preferred method for installing a second portion of the implant to the posterior surface of the sternum.

FIG. 12 is another view of the method of FIG. 11.

FIG. 13 shows the implant according to the present invention installed on the posterior surface of the sternum.

FIG. 14 is a view of the underside of an embodiment of the platform according to the present invention.

FIG. 15 is a view of the top of the platform of FIG. 14.

FIG. 16 is a side view of another embodiment of the platform of the present invention.

FIG. 17 is a side of another embodiment of the platform of the present invention.

FIG. 18A is an anterior view of the human spine.

FIG. 18B is a lateral view of the human spine.

FIG. 19A-D illustrate various abnormal curvatures of the spine due to scoliosis.

FIG. 20 illustrates abnormal rotation of the vertebrae of the spine as a result of scoliosis.

FIG. 21 illustrates another embodiment of the invention for treating scoliosis.

FIG. 22 illustrates an alternative embodiment for delivering a pulsed magnetic field to a body member.

FIG. 23 is a schematic view of an alternative embodiment for delivering a repulsive force to a body member.

FIG. 24 is a schematic view of the device of FIG. 23 with a fluid pump.

FIG. 25 illustrates an alternative embodiment of a repulsion device incorporating a mechanical jackscrew.

FIG. 26 illustrates an alternative embodiment of a repulsion device incorporating an electric jackscrew.

FIG. 27 A-E illustrate schematic diagrams of various forms of esophageal atresia.

FIG. 28 shows a system for elongating the esophagus in accordance with the present invention.

FIG. 29A illustrates a pneumatic system for incrementally adjusting the location of an internal magnet.

FIG. 29B illustrates an internal electromagnet in accordance with the present invention.

FIG. 30 illustrates a reformation device for altering the physical appearance of a body part in accordance with the present invention.

FIG. 31 illustrates a side view of the anatomical features of a human nose.

FIG. 32A shows a side view of a patient before undergoing the corrective procedure of the present invention.

FIG. 32B shows a side view of a patient of FIG. 32A after undergoing the corrective procedure of the present invention.

FIG. 33A illustrates injection of a magnetic solution in accordance with the present invention.

FIG. 33B illustrates the magnetic particles of the solution of FIG. 33A being manipulated by an external magnet.

FIG. 34 illustrates an magnetic jewelry clasp in accordance with the present invention.

FIG. 35A illustrates a side view of the magnetic jewelry clasp of FIG. 34A.

FIG. 35B illustrates a side view of an another embodiment of magnetic jewelry clasp mounted on the inside of the ear lobe.

FIG. 35C illustrates a side view of an another embodiment of magnetic jewelry clasp mounted on the inside and outside of the ear lobe.

FIG. 36 illustrates the magnetic jewelry clasp of FIG. 34 and resulting compression of the ear lobe.

FIG. 37 illustrates another embodiment of the magnetic jewelry clasp in accordance with the present invention.

FIGS. 38 and 39A illustrate the magnetic jewelry clasp of FIG. 37 mounted on the ear.

FIG. 39B illustrates an alternative embodiment of the magnetic jewelry clasp of FIG. 37 mounted on the inside and outside surfaces of the earlobe.

FIG. 40 illustrates a cross-section of human skin.

### DETAILED DESCRIPTION OF THE INVENTION

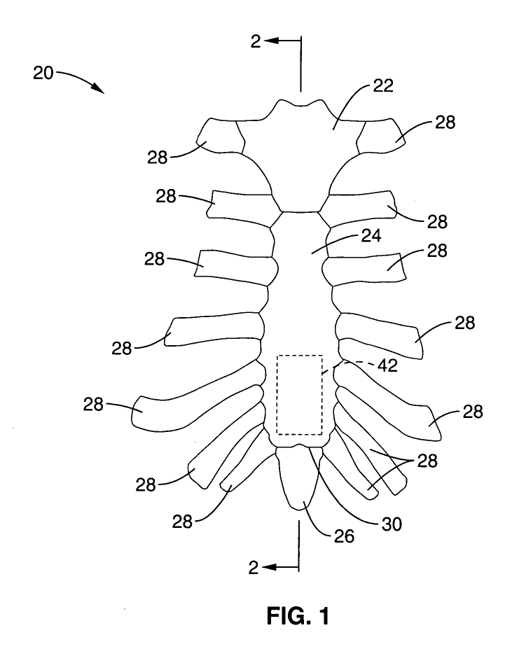

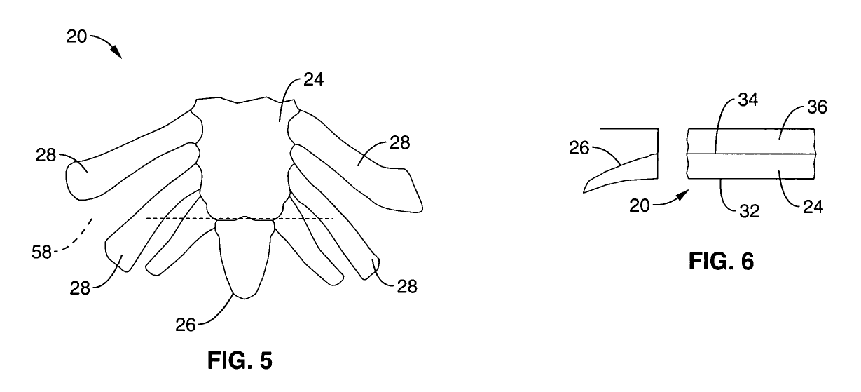

Referring more specifically to the drawings, for illustrative purposes the present invention is embodied in the apparatus an methods generally shown in FIG. 1 through FIG. 17 and FIGS. 21-26 and 28-40. It will be appreciated that the apparatus may vary as to configuration and as to details of the parts, and that the methods may vary as to the specific steps and sequence, without departing from the basic concepts as disclosed herein.

The present invention utilizes a system in which a small magnet is implanted in cooperation with an internal body member to apply a corrective force to the body member by virtue of its attraction to an adjustable magnet in an external device that is comfortable and cosmetically pleasing.

Small rare earth metal magnets can produce considerable force and can be manipulated in terms of size, shape and position. This force can be used to alter growth and development of skeletal structure and soft tissue. The biology of tissue response to force has been well studied. Clinical application of this powerful biologic principle has been limited by the difficulties of applying force through external bracing or through internal pins manipulated by external devices (e.g., bone lengthening through distraction osteogenesis). Magnetic force fields can be used to apply force to implanted magnets attached to an internal structure without violating the skin and soft tissue. The magnetic force field can be manipulated externally to adjust the direction, strength and speed at which the deformity is corrected.

1. Pectus Excavetum

FIGS. 1-16 illustrate a preferred embodiment of the invention relating to the correction of pectus excavetum. FIG. 1 illustrates a schematic, anterior view of a human sternum 20. The sternum 20 is an elongated, flatted bone, forming the middle portion of the anterior wall of the thorax. The sternum 20 generally consists of three parts: the manubrium 22, which at its upper end supports the clavicles (not shown); the body or gladiolis 24, which interfaces at its upper end with the lower end of the manubrium 22, and the xiphoid process 26, which interfaces at its upper end with the lower end of the gladiolis 24 at junction 30. The margins of sternum 20 articulate with the first of seven pairs of ribs 28.

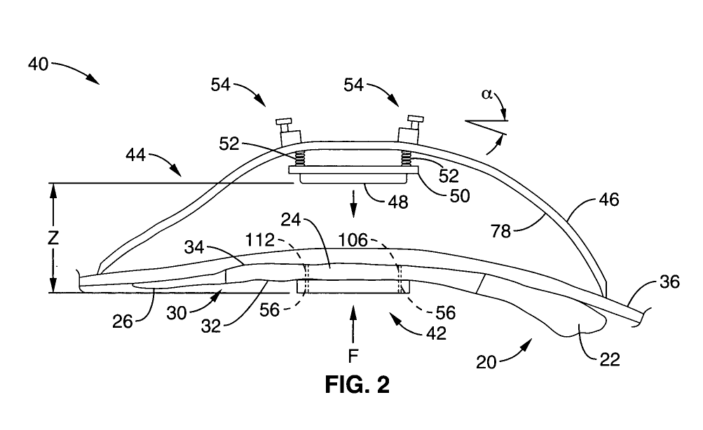

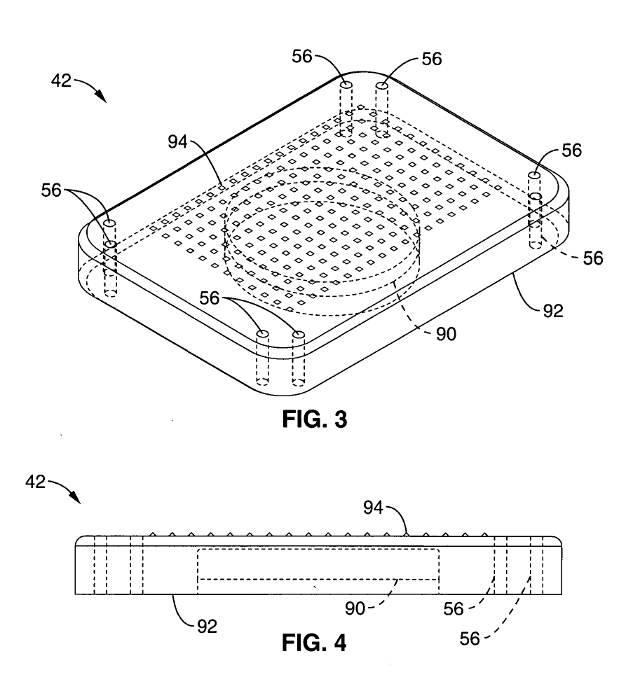

As shown in FIG. 1 and illustrated as a cross-sectional view of a corrected patient's chest in FIG. 2, a magnetic substernal implant 42 may be installed on the posterior surface 32 the body 24 of the sternum 20, just above the xiphoid process 26. Illustrated in greater detail in FIG. 3, the implant 42 preferably comprises a rare earth magnet 90, or an array of rare earth magnets housed in casing 92. The casing 92 may comprise any biocompatible material such as medical grade epoxy, titanium or suitable material used in the art. Casing 92 preferably has mounting holes 56 for fixation at each corner. The casing 92 may also have a plurality of protrusions 94 to enhance the attachment of the implant 42 with the sternum 20.

The magnetic implant 42 is sized to fit comfortably behind the sternum.

An exemplary implant may 3 inches long, 2½ inches wide and 3/16 thick.

However, the size of the implant may vary according to patient anatomy.

FIGS. 2 and FIGS. 5-13 illustrate an exemplary method of surgically installing the implant 42. A 3 cm substernal transverse incision 58 is made through the patient's skin 36. The ziphoid process 26 is then separated from the lower sternum body 24 and a pocket is bluntly dissected behind the posterior surface 32 of the sternum, as illustrated in FIG. 6.

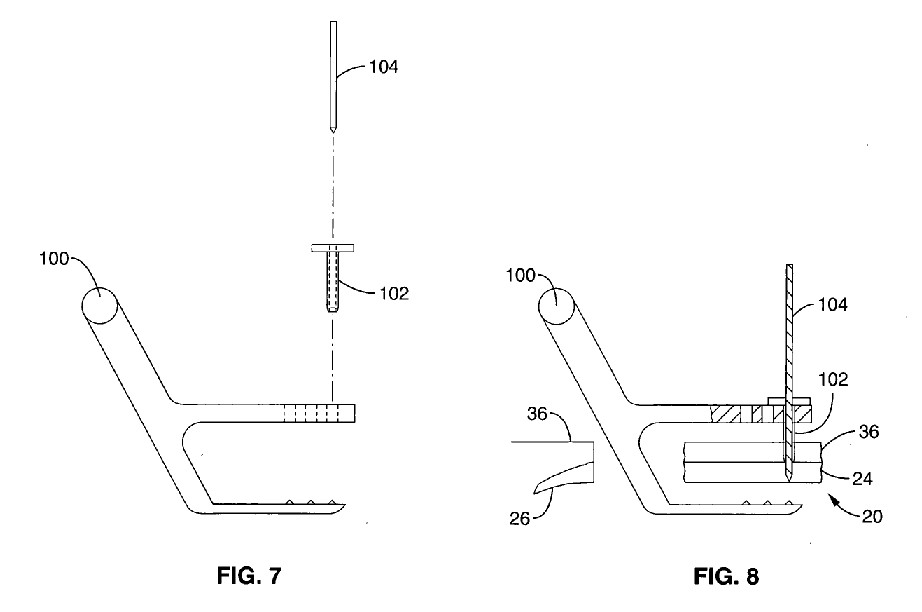

The implant 42 is attached to the posterior surface of the sternum with sutury passed through the holes 112 in the sternum illustrated in FIG. 2. Using laparoscopic or arthroscopic visualization, a drill guide 100 is inserted and positioned over the proximal end of the sternum body 24, as shown in FIGS. 7 and 8. A small stab wound is made into skin 36, and the drill sleeve 102 is inserted through the guide 100. The sternum 20 is then drilled under direct visualization to bore one or more distal bores 106 from the anterior surface 34 of the sternum through to the posterior surface 32. Distal bores 106 preferably line up with the corresponding mounting holes 56 in casing 92.

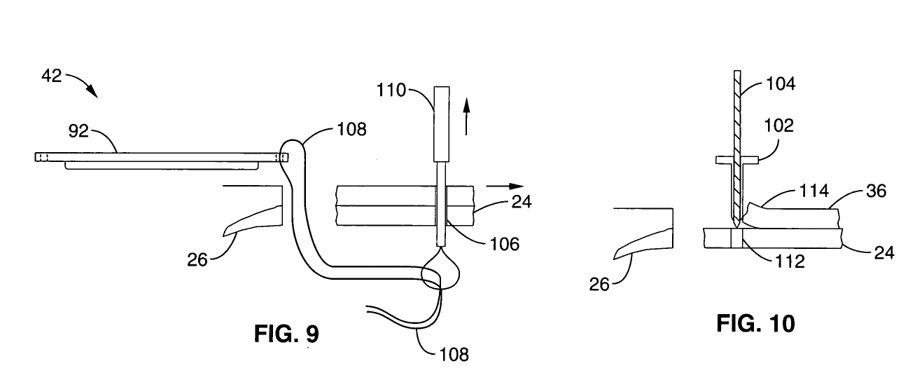

Referring now to FIG. 9, a distal suture 108 is looped through one of the mount holes 56 of casing 92. The distal suture 108 preferably comprises a heavy braided suture commonly used in the art, e.g. #2 or #5 ticron. The suture is then fed under the sternum 20 and a suture retriever 110, such as a Hewson type, is used to pull the distal suturel 08 through the corresponding distal bore 106 in the sternum body 24 and the anterior skin stab wound. The process is repeated for the second corner of the distal end of the casing 92.

Referring now to FIG. 10, the proximal end 114 of skin 36 and subcutaneous tissues from the anterior sternum proximal to the sterno-xiphoid junction 58 are pulled back to expose the proximal end of the sternum body 24. The drill guide 100 is moved transversely along the sternum body 24 to the exposed portion of the sternum under proximal end 114. Once sufficient exposure is obtained, one or more proximal bores 112 are drilled under direct vision through the sternum anteriorly-to-posteriorly, thus providing anchoring points for all corners of the casing 92.

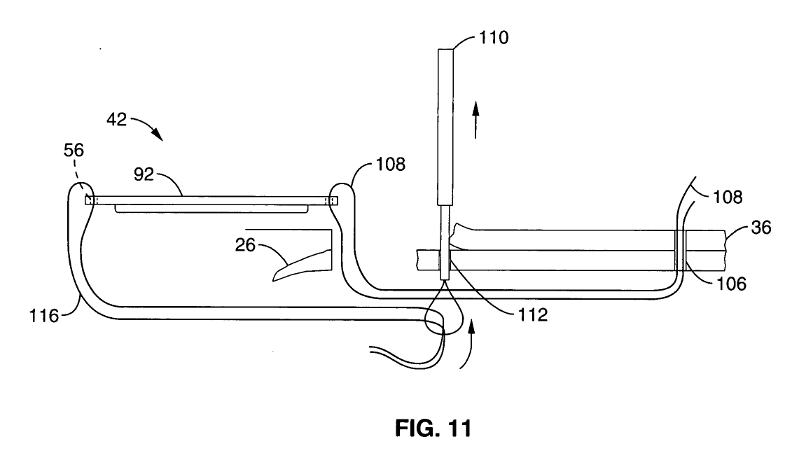

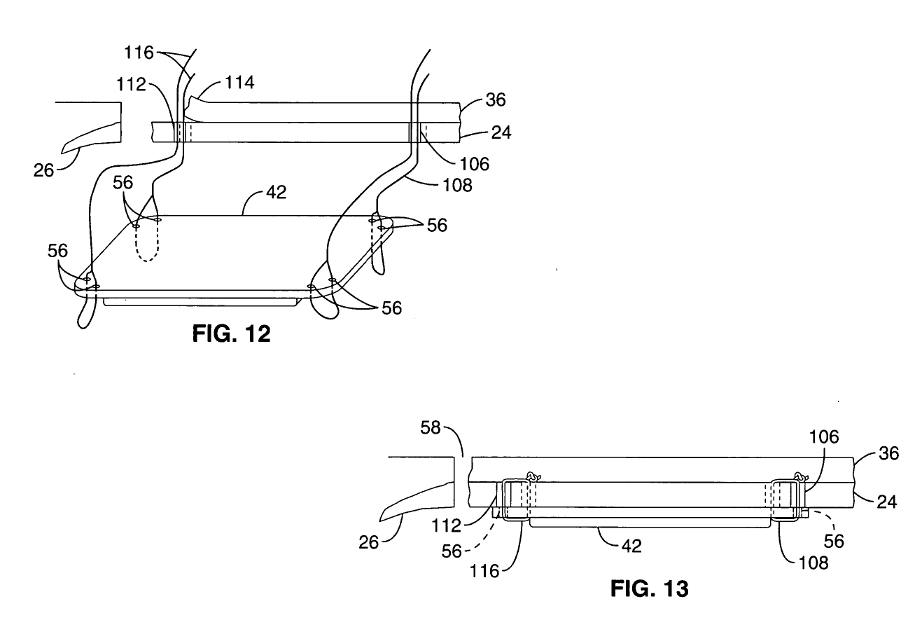

Now referring to FIGS. 11 and 12, a proximal suture 116 is looped through one of the mount holes 56 on the proximal end of casing 92. The suture is then fed under the sternum 20 and suture retriever 110 is used to pull both ends of the proximal suture 116 through the corresponding proximal bore 112 in the sternum body 24. The process is repeated for the second corner of the proximal end of the casing 92.

As seen in FIG. 12, both sets of proximal and distal sutures 116,108 are pulled to guide the implant 42 up behind the sternum 20 and maintain the apposition of the casing 92 to the sternum 20 with traction on the sutures.

Referring now to FIG. 13, the proximal sutures 116 are tied down firmly over the sternal bone bridge to secure the implant 20 to the proximal surface 32 of the sternum. Under direct vision, the process is repeated for the distal sutures 108.

Although FIGS. 9-13 illustrate a preferred embodiment using suture to fasten the implant 42 to the sternum 20, it is appreciated that any number of different fastening means commonly known in the art may be used to secure the implant 42. For example, bolts (not shown) may be passed through bores 106, 112, threaded into threaded mounting holes 56 of casing 92 and torqued down to secure the implant 42 to the posterior surface 32 of the sternum 20. Alternatively, the implant may be fastened to either the posterior or anterior sides of the sternum via cables that wrap around the sternum. In this configuration, since the implant is closer to the platform, the internal magnetic member may be any magnetically responsive material, such as an iron plate with biocompatible coating (e.g. titanium).

Surgical placement generally requires a brief outpatient general anesthesia. The procedure takes about 30 minutes and requires minimal post-operative analgesia.

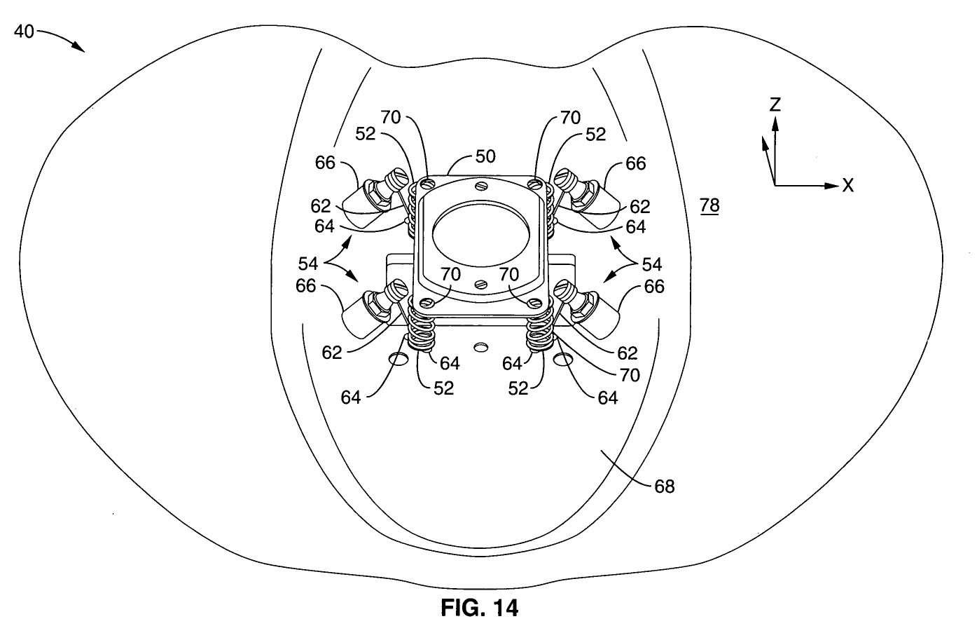

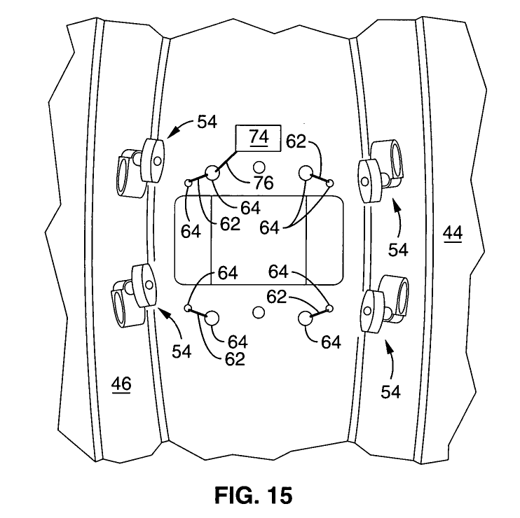

FIGS. 2 and 14-17 illustrate several embodiments of an external magnet platform of the present invention for treating pectus excavatum. FIGS. 14 and 15 show an embodiment having a platform 40 configured to be worn over a patient's chest. Platform 40 comprises a chest plate 44 sized according to the patient's anatomy. Generally, a mold is made of the individual's chest deformity. From this the desired end point position of the sternum and chest wall shape are molded to create the chest plate 44. FIG. 14 is a bottom view of platform 40, showing the underside 78 of chest plate 44. In addition to being contoured to comfortably rest on the patient's chest, the underside 78 of the chest plate is cut away to create cavity 68 that allows the chest to expand outward as a result of treatment.

In a first configuration, an external magnet 48 is hung from the underside 78 of the chest plate 44 by a plurality of adjustment cables 62. External magnet 48 is preferably a rare earth magnet, or array of rare earth magnets. The external magnet has an adjustable stage, or mounting plate 50, which has a plurality of holes 70 to secure cables 62. As illustrated in FIGS. 14 and 15, the magnet 48 is hung with 4 cables. However, other configurations, such as a three cable design (not shown), may also be used. The cables 62 are coupled to the chest plate via adjustment members 54. Cables 62 lead from the magnet plate 50 out to the exterior surface 46 and back through to the underside of the chest plate via through holes 64 to terminate at adjustment member 54. One or more biasing springs 52 may be imposed between the chest plate 44 and the magnet 48, creating a tensile force on cables 62 so that the magnet is biased to the furthest orientation away from the chest plate 44 that is allowed from the cables' length.

By turning adjustment member 54 from the top of the chest plate illustrated in FIG. 15, the cable 62 may be shortened, thereby advancing one corner of the magnet plate 50 upward toward the chest plate 44. By rotating the adjustment member in the opposite direction, the cable is extended, thereby advancing one corner of the magnet plate 50 toward from the patient's chest and away from the chest plate 44. When all the adjustment members are moved the same increment, the magnet will translate toward or away from the patient's chest in the Z axis (see FIGS. 2 and 14). The magnet may also be rotated angle θ about the X or Y axis by manipulating the adjustment members 54 to lengthen or shorten one or more cables 62 with respect to the remaining cables.

The external magnet 48 and the implant magnet 90 are configured so that their opposite poles face each other, thereby generating an attractive force between the two magnets. By manipulating the distance of the external magnet 48 from the chest plate 44 in the Z direction, the amount of force applied to the internal magnet can be incrementally tuned or adjusted. By manipulating the orientation of the external magnet 48 with respect to the chest plate 44 in the X and Y directions, the direction of force applied to the internal magnet can be incrementally adjusted.

The chest plate 44 is preferably comprised of a rigid material, such as a rigid thermoplastic or polymer or steel reinforced polymer that does not deform as a result of the magnetic forces, allowing external magnet 48 to remain stationary with respect to the patient's chest. As a result of the constant force applied from the external magnet 48, the implant 42 imposes a corrective outward force F on the posterior surface 32 of the sternum 20. This outward force incrementally repositions/deforms the sternum 20 to move outward from the patient's chest cavity. By adjusting the angle of the external magnet in the X and Y directions, the force generated on the implant 42 may be directed to orient the sternum in the X and Y axes as well to correct asymmetric lesions.

An initial adjustment of the platform is made after the implant is placed in the outpatient surgical procedure. When the sternum 20 and implant 42 move toward the external magnet 48, the force generated between the magnets increases. If this force becomes too great and becomes uncomfortable for the patient, the magnet may be retracted toward the chest plate 44, thereby returning the magnetic force to the optimum comfort level for the patient. This process may be repeated for a number of intermediary steps, until the sternum 20 is gradually repositioned and/or deformed toward the desired final position and orientation.

The platform 40 may also include a strain gauge 74, or other force measuring means, to accurately determine the force being generated by the magnets. Strain gauge 74 may be connected via lead wires 76 to various points on the magnet plate 50 so that the pressure on each quadrant of the magnet may be accurately assessed. Strain gauge 74 may also comprise an LCD display (not shown) so that the patient or physician may readily assess whether the external magnet 48 is properly oriented, and adjust the magnet if need be.

The platform 40 is held in place by the magnetic pull between the two magnets, and in addition may be secured in place with a loose elastic band (not shown) around the chest. The principal force holding the platform 40 in place is the magnetic field itself. The patient may adjust the platform 40 to comfort and thus ensure against pressure damage to soft tissue. The patient may be taught to how to manipulate the external magnet 48 up and down to adjust and balance the force pulling the sternum 20 outward.

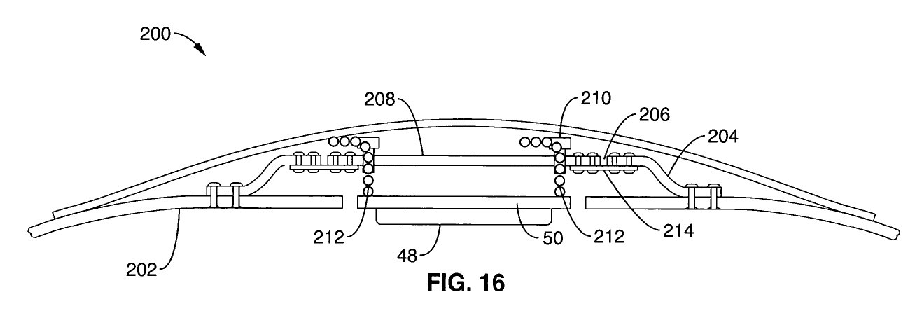

To provide extra comfort to the patient, and prevent the any unwanted manipulation of the adjustment members, a cover, such as that shown in FIG. 16, may be provided to cover the chest plate while the platform is being worn.

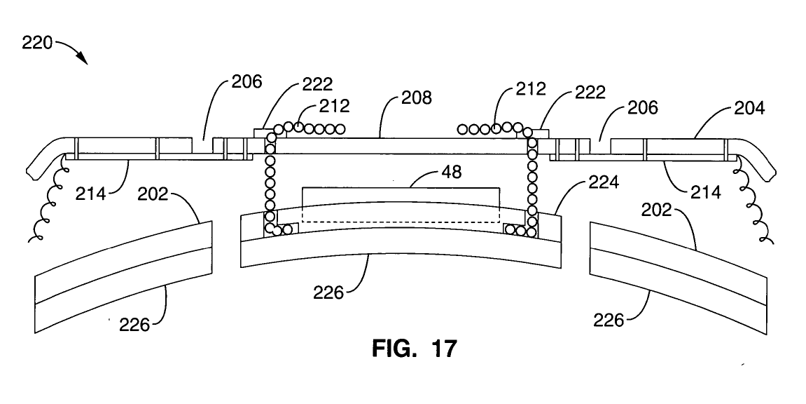

A preferred embodiment of the invention incorporating a bridged platform 200 is illustrated in FIGS. 16 and 17. Platform 200 has a chest plate 202 having a support 204 with opening 206 at it center. Chest plate 202 and support 204 may be separate pieces fastened together as shown in FIG. 16, or one integrated piece (not shown). Load member 208 is positioned in the opening 206 of support 204, and is bridged by a plurality of thin beam force sensors 214.

Load member 208 has a plurality of adjustment members 210 that retain magnet plate 50 and magnet 48 via a hanging means 212. Adjustment member 210 comprises an in-line screw, such as a jack-screw, lead screw, ball screw, or the like, which is hollowed out to support hanging means 212. As shown in FIGS. 16 and 17, hanging means 212 comprises a ball chain, but may also comprise a cable, wire, or the like. Alternatively, adjustment members 210 may comprise extended screws (not shown) that terminate a ball joint in magnet plate 50.

Adjustment members 210 may be manipulated to lower or raise the magnet 48, or adjust the angle of the magnet, as described in the embodiment of FIGS. 14 and 15. By turning screw 210 clockwise, one quadrant of the external magnet 48 may be precisely lowered to change the angle of the external magnet 48 with respect to the patient's chest, thereby changing the direction of the force applied to the implant 42. By turning all the screws the same clockwise increment, the magnet is lowered to generate a larger attractive force on the implant. Correspondingly, counter-clockwise rotation raises the external magnet to lower the attractive force on the implant 42.

When the platform is placed against the patient's chest, the attractive force between the implant 42 and the external magnet generates a load on load member 208. This load is sensed at all four quadrants by the thin beam force sensors 214. Readings from the sensors 214 are received by a force measuring means, such as the strain gauge 74 illustrated in FIG. 15, to provide accurate data on the force applied at each quadrant of the external magnet. This enables the treating physician or patient to accurately assess corrective the force being applied to the sternum, and modify the force if not at the desired level.

FIG. 17 illustrates an alternative embodiment having a platform 220 wherein the adjustment member comprises a clasp 222 for incrementally adjusting the extended length of ball chain 212, which is attached to each corner of the external magnet cradle 224. By changing the position at which the clasp 222 engages the ball chain 212 (similar to adjusting a necklace of bracelet), the height at any one quadrant of the magnet 48 may be changed with respect to the patient's chest to vary the force or direction of the corrective magnetic field. Chest plate 202 and cradle 204 may also have a layer of padding 226 to provide further comfort for the patient.

Over time, the steady gradual force applied to the sternum stretches the ligaments connecting the sternum 20 to the ribs. The sternum 20 itself may also deform as a result of the magnetic forces. The result is a reoriented and/or repositioned sternum without the characteristic depression of the pectus excavatum deformity.

As the sternum 20 moves closer to the external magnet 48, the patient or physician will typically readjust the position of external magnet 48 farther up into the chest plate. This is easily accomplished by adjusting the length of the four ball chains that suspend the magnet cradle 224.

2. Scoliosis

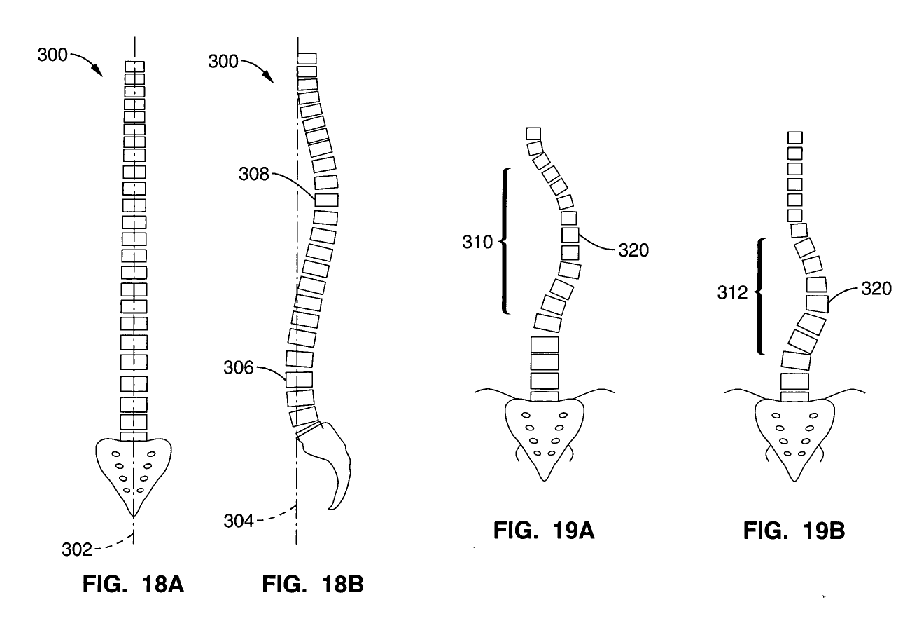

FIGS. 18A and 18B illustrate the curvature of a normal spine 300. The spine is relatively straight in the sagittal plane 302 and has a double curve in the coronal plane 304. As shown below, the thoracic section 308 of the spine is convex posteriorly and the lumbar section 306 of the spine is convex anteriorly. Normally there should be no lateral curvature of the spine about the saggital plane 302.

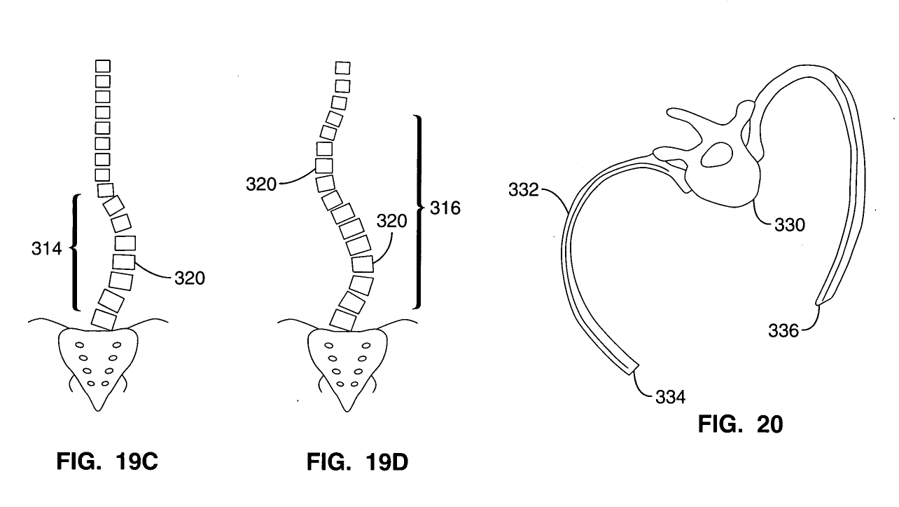

Scoliosis is a deformity that generally comprises by both lateral curvature and vertebral rotation. FIGS. 19A-D illustrate various forms of abnormal lateral curvature of the spine. FIG. 19A shows abnormal thoracic curvature 310. FIG. 19B shows abnormal thoracolumbar curvature 312. FIG. 19C shows abnormal lumbar curvature 314. Finally, some cases involve a double curvature of the spine, as shown in FIG. 19D shows abnormal thoracic curvature.

FIG. 20 illustrates rotation of the spine and corresponding effect on the rib cage 332 s a result of scoliosis. As the disease progresses, the vertebrae 330 and spinous processes in the area of the major curve rotate toward the concavity of the curve. As the vertebral bodies rotate, the spinous processes deviate more and more to the concave side and the ribs follow the rotation of the vertebrae. The posterior ribs on the convex side 336 are pushed posteriorly, causing narrowing of the thoracic cage and the characteristic rib hump seen in thoracic scoliosis. The anterior ribs on the concave side 334 are pushed laterally and anteriorly.

Now referring to FIG. 21, a schematic view of external platform 350 is illustrated with implant 340 installed on vertebrae 330 of the spine. Vertebrae 330 is preferably located at the apex 320 of the abnormal curvature shown in FIGS. 19A-D. In a preferred embodiment implant 340 is anchored to vertebrae 330 via a bone screw 336. Screw 336 may be threaded into a bore 334 in the pedicle 332 of the vertebrae according to commonly used procedures for a variety of spinal conditions, including degenerative disc disease and scoliosis. Examples of such systems are disclosed in U.S. Pat. Nos. 6,648,915; 6,010,503; 5,946,760; 5,863,293; 4,653,481, etc., the entire disclosures of which are incorporated herein by reference.

Once pedicle screw 336 is installed, internal magnet 342 may be fastened to screw 336 via magnet casing 338 and nut 346. Following the same procedure, a second internal magnet 344 may also be installed on the pedicle on the opposite side of implant 340.

After installation of implant 340, external platform 350 may be placed on the patient's back 366 adjacent to the installed implant. Platform may be retained to the torso of the patient by a strap the circles the patient's waist or chest at the elevation of the implanted vertebrae 330. Platform 350 comprises a support 352 that adjustably holds first external magnet 360. First external magnet 360 is hung inside recess 364 by a plurality rods 354, which are fastened to external mounting plate 358 housing magnet 360. The angle and height of magnet 360 may be incrementally adjusted by adjustment member 356.

As illustrated in FIG. 21, external magnet 360 and internal magnet 342 may be positioned with facing positive poles (or facing negative poles) to generate a repulsive force between the platform 350 and the implant 340. The resulting magnetic force creates a rotational moment R on the vertebrae 330 to incrementally reorient the vertebrae 330 and diminish the abnormal rotation angle β. As vertebrae 330 rotates to a more normal orientation, the rest of the vertebrae of the spine follow.

If a second internal magnet 344 is installed opposite internal magnet 342, a second external magnet 362 may be positioned opposite internal magnet 344. As shown in FIG. 1, the opposing magnets may be positioned to generate an attractive force, thereby increasing the magnitude of the rotational moment R on the vertebrae.

In addition to effecting rotation of the spine, platform 350 may be oriented to correct for lateral curvature of the spine. By placing the platform 350 to the line up to the left of the implants, as shown in FIG. 21, a translational force T is created on the vertebrae 330 as a result of the attractive force between the second external magnet 362 and second internal magnet 344. In this configuration, external magnet 360 may be removed to increase the attractive force. The platform 350 may be incrementally repositioned to continue translation of the vertebrae 330.

3. Other Applications

Variations of the above embodiments could be use to gradually correct a variety of deformities. For example, pectus carinatum (a deformity of the chest involving a sternal protrusion) may be treated with the embodiments shown in FIGS. 14-17 and orienting the magnets to apply a repulsive rather than attractive force.

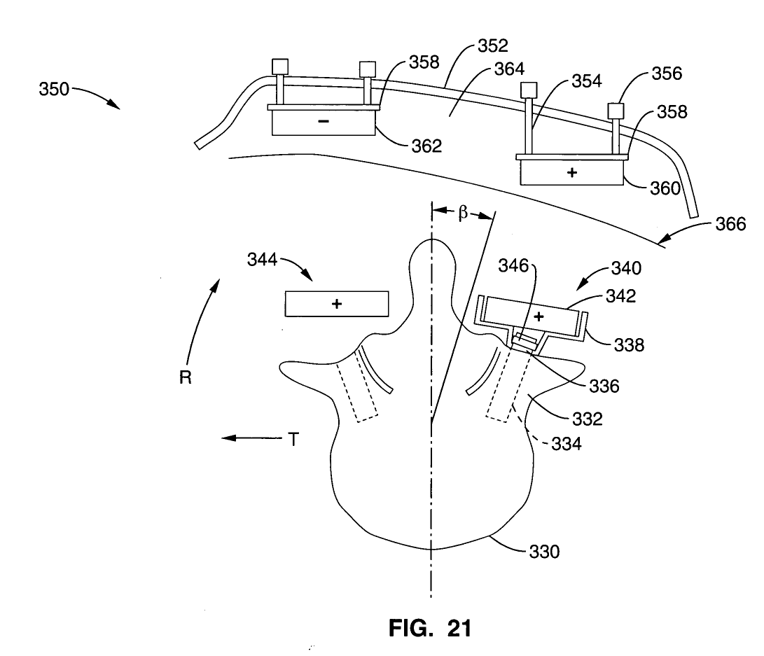

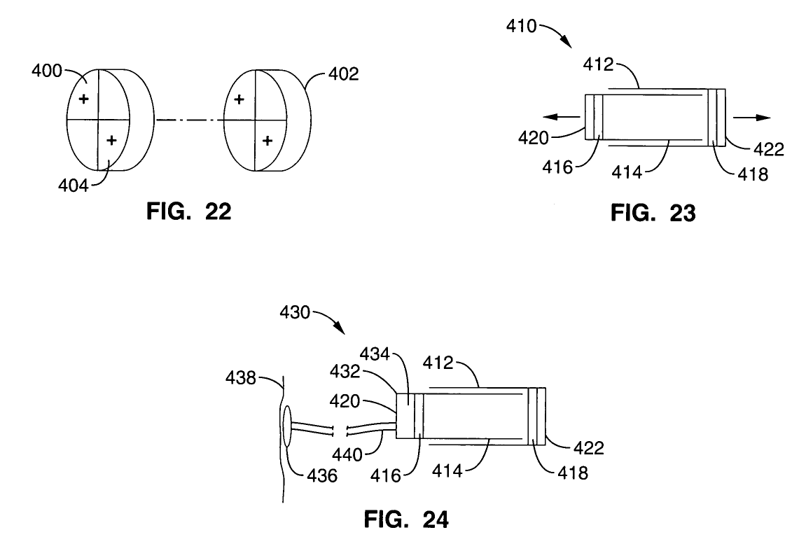

In another alternative embodiment, which may be beneficial for soft tissue deformities, a magnetic force discontinuously applied in order to accommodate blood flow to the tissue. For example, the force may be applied for a period of time (e.g. a minute) and then taken off for another period of time (applied in a pulsed fashion) in order to let blood flow back to the tissue being “reformed”. In one embodiment illustrated in FIG. 22, a pulsed force field is generated by rotation of the external magnet 402 with respect to fixed internal magnet 402. The magnets may have magnetized quadrants 404 that repel/attract or become neutral upon a 90 degree rotation with respect to each other to achieve tension alternating with relaxation. In an alternative embodiment, the external magnet is moved closer and then farther from the internal magnet by rotating it on a cam (not shown).

In addition to magnetic force fields configured to manipulate body members by attraction of two magnets (e.g. the device above for repair of pectus excavatum), the magnets may be configured to provide a repulsive force (e.g. a magnetic Elizeroff to lengthen bone). In the embodiment illustrated in FIG. 23, internal repulsion device 410 comprises first member 414 partially encased in second member 412, wherein first member 414 is allowed to slide inside second member 412. Each member has a corresponding internal magnet 416, 418 which are configured to repel each other, thus forcing first member 414 to separate from second member 412 to form a “magnetic spring” to distance anatomy located on ends 420 and 422. The repulsive force may be varied by adjusting the position of magnets 418 and 416 away from ends 420 and 422.

Repulsion device 410 may be used in a variety of applications where gradual force may be applied to reposition or deform one or more body members. For example, device 410 may be disposed such that ends 420 and 422 are attached to two separate locations of a bone to lengthen or alter the shape of the bone.

In an alternative embodiment illustrated in FIG. 24, repulsion device 430 may be used having reservoir 434 and pump 436. Pump 436 may be positioned underneath the patient's skin 438, such that fluid may be directed through lead line 440 to reservoir 434 in second chamber 432. The pump may be used to increase the volume of reservoir 434, thereby distancing magnet 416 away from end 420 to incrementally increase the repulsive force between 416 and 418.

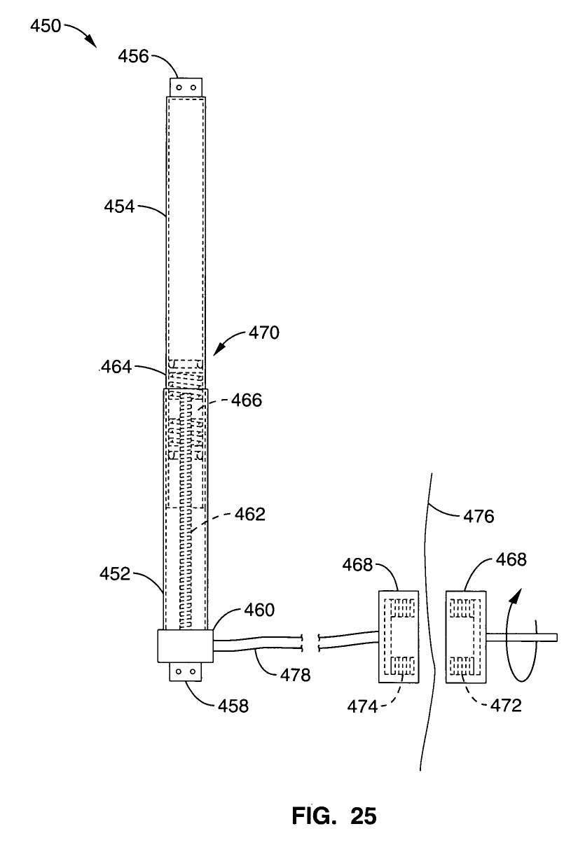

In another alternative embodiment illustrated in FIG. 25, repulsion device 450 comprises a mechanical jackscrew 470. The device has a first member 452 and second member 454 that apply a repulsive force to attachment points 456 and 458 that may be attached to one or more body members. Rotary magnet coupling 468 has an internal magnet 474 under the patient's skin 476 and a corresponding external magnet 472.

The magnets are polarized such that rotation of the external magnet 472 causes a proportional rotation in external magnet 474, which in turn rotates flexible shaft 478. Rotation of flexible shaft 478 is transferred to rotation of screw 462 located on first member 452 via worm gear 460. Nut 466 is attached to second member 454 and is threaded to screw 462 such that rotation of screw 462 causes the first member 452 to separate from 454. Additional force and separation may be achieved by further rotation of external magnet 474. Springs 464 may optionally be employed to create an additional preload between the first and second members.

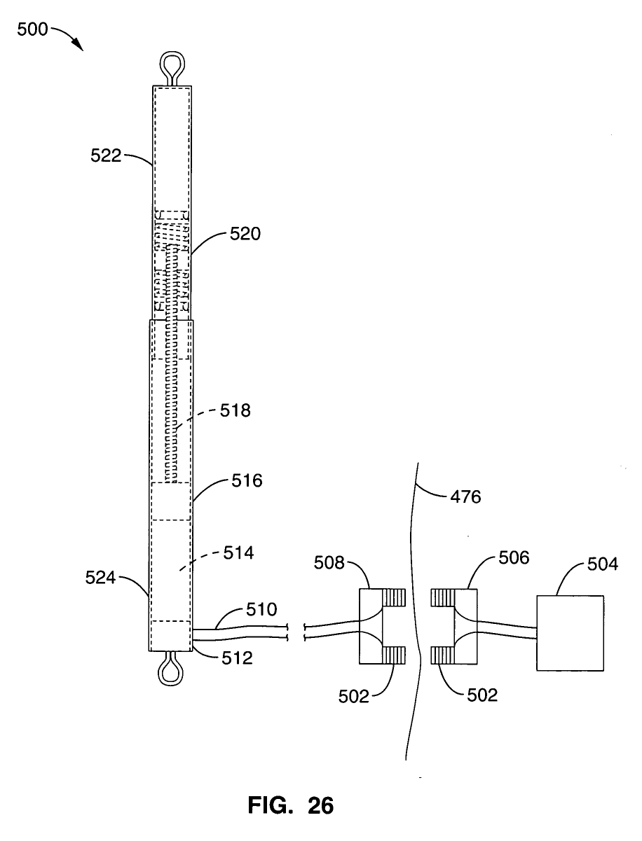

FIG. 26 illustrates another alternative embodiment of a repulsion device 500 having an electric jackscrew. Control box 504 controls rotation of magnetic coupling 502. A signal is sent via wire 510 to electronics 512 to control electric motor 514, which drives rotation of screw 518 through gear reduction 516. Thus, a repulsive force may be incrementally applied to separate first member 524 from second member 522.

4. Esopahgeal Atresia

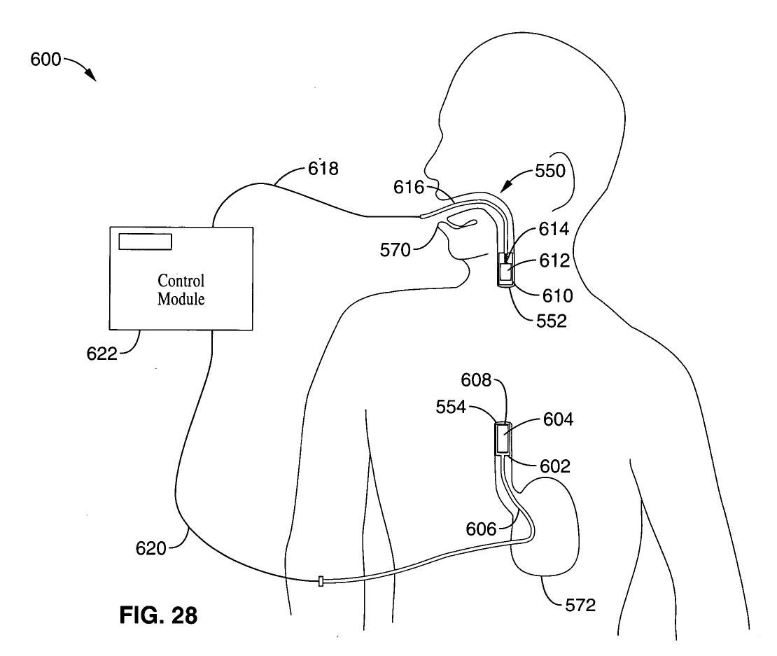

Referring to FIG. 28, a lumen lengthening system 600 for elongating the esophagus in children born with long-gap esophageal atresia is disclosed. The system 600 uses magnetic attraction between magnetic members put in the upper and lower ends of an esophageal atresia to gradually allow the tissue to grow and elongate until enough length has been achieved to reattach the ends.

As shown in FIG. 28, an incision is made in the patient's abdomen and through the first magnetic member 604 is fed into the patient's stomach 572 and into the lower esophagus 554 via a catheter tube 606. Catheter tube 606 generally comprises flexible tubing such as i.v. tubing or the like, and is of sufficient length to deliver the first magnetic member 604 through the abdomen to the upper extent of the lower esophageal atresia 554.

A laparoscopic approach may be used to minimize the incisions and patient morbidity. The first magnet 604 is encased in a biocompatible tube 602 that is shaped to fit within the lumen of the esophageal passageway 550 tube 602 may comprise titanium or other suitable biocompatible material.

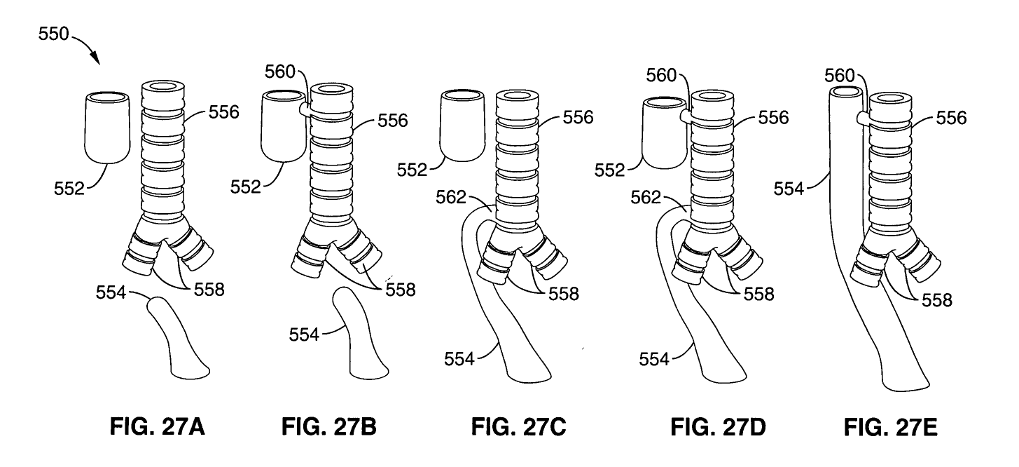

Referring further to FIG. 28, a second magnetic member 612 is inserted into the esophagus 550 via tubing 616 that is feed into the patient's mouth 570. If a TEF is present as shown in FIG. 27B, the fistula 562 is first detached and the hole in to the trachea 556 repaired. The second magnetic member 612 is encased in biocompatible container 610 that is shaped to be delivered to the lowest extent of the upper esophageal atresia 552. The first and second magnetic members 604, 612 are configured to generate an attractive force between each other, thereby impinging on the ends of the lower and upper esophageal sections 552, 554 to place the esophageal lumens in tension and slowly lengthen the esophageal segments toward each other.

In the embodiment illustrated in FIG. 28, the location of the second internal magnet 612 may be adjusted in relation to the canister 610 and the lower magnet 604, which is relatively fixed. Actuation of the second internal magnet 612 may be facilitated by a linear motion mechanism 614, such as a stepper motor and lead screw, or linear actuator commonly used in the art. The linear motion mechanism 614 may be connected to a control module 622 via wiring 618 that runs along the central lumen of catheter tube 616.

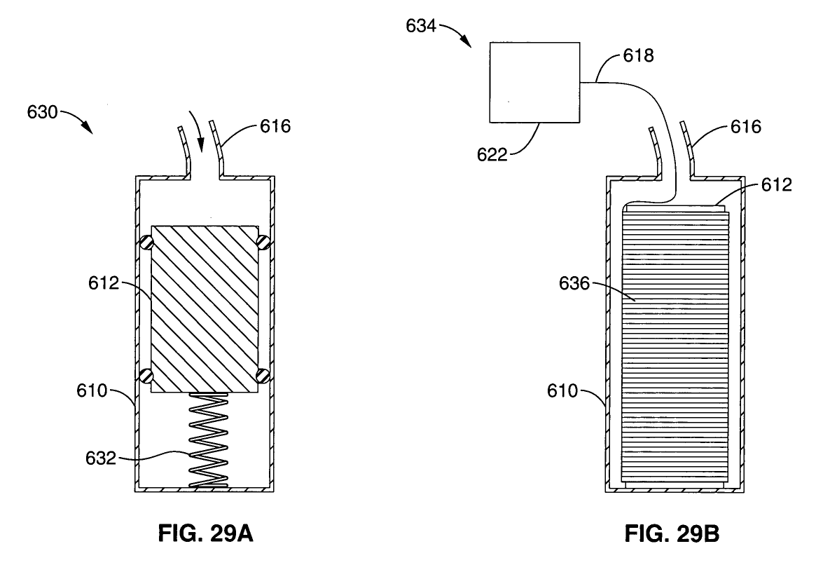

Referring to FIG. 29A, the magnet location may also be adjusted via a pneumatic system 630 wherein air is pumped into the container 610 via tubing 616 to facilitate a pressure on the magnet 612 to force the magnet down the tube against a biasing spring 632.

In an alternative embodiment shown in FIG. 29B, an electromagnetic system 634 may be incorporated. In this embodiment, the second magnet 612 comprises a cylindrical magnet wrapped with wiring 636. The wire 636 comes out along the tube 616 via wiring 618 and is hooked up to a control module 622. Then, by changing the electrical current to magnet 612, the force with which the first and second magnets are attracted to each other can be varied, and the polarity can be reversed so that the tension can be relaxed.

Referring back to FIG. 28, the first internal magnet 604 may also be coupled to a pressure sensor, such as a flexible membrane 608, for measuring the amount of force being applied by the magnetic attraction between the two magnets. The pressure sensor 608 may be wired out to the via wiring 620 to the control module 622. Control module 622 may be configured to shut automatically and incrementally advance or retract second magnet 612 (increase/decrease pressure to system 630 of FIG. 29A, or turn on/off or increase/decrease electrical current to device 634 of FIG. 29B) according to the desired attractive force between the magnets. The control module 622 may also be configured to retract the second magnet 612( by reversing actuation of motor 614 or releasing pressure in system 630) when the force becomes too great, and thus limit tissue damage. The control module 622 may further be configured allow the second magnet to cycle, i.e. vary the position of the magnet over time to have an attractive force to stretch the tissue gradually until it reaches a certain pressure or period of time, and then back out to relieve the pressure for some period of time. The system 600 takes a break as the tissue of the esophageal lumens, no longer under stretch, has a chance to renew its blood supply and then recycle again. It is appreciated that the moveable magnet may also be placed in the lower esophageal segment 554, and a corresponding fixed magnet in the upper esophageal segment 552.

In an alternative embodiment (not shown), magnetic members 608 and 612 comprise a series of small magnets that are stacked on either end of the esophagus according to the desired attractive force and distance between the ends of the lower and upper esophageal atresias. The attractive force can be modified by simply adding or subtracting magnets to containers 602 and 610.

The above devices and methods are advantageous in that the two ends of the esophagus are gradually drawn directly to each. Eventually, when they are very close together, a strong enough force may be applied at the end to auto-anastomose the esophageal tissue, i.e. necrose the tissue out in between.

Although the description above devices and methods are directed to lengthening of the esophagus, the subject matter disclosed in FIGS. 28-29 may be applied to elongate a number of lumens or cavities located in the body, such as the urethra, vaginal atresia, or to lengthen two sections of the intestine for patients have short-bowel syndrome.

5. Cosmetic Surgery

The concepts previously described for reformation of defects may also be used to do procedures usually requiring plastic surgery or cosmetic surgery. Using the same principle with different sized magnets, a nose, an ear, a cheekbone, or other body part may be reshaped using an implanted magnet and external form with a magnet attached. In general, a patient could choose exactly how they wanted a particular body part to look like, for instance, the shape of a nose. The shape could be made as a mold or a form, and the patient would choose exactly how big or little or sharp or narrow it would be in advance. Then, the appropriate magnet or magnets are implanted in that body part and subsequent appropriate magnetic force is applied on the form to essentially mold the body part to the pre-made form over time. This could be used for cosmetic procedures on various parts of the body including cartilaginous and bony tissue. It could be used to raise depressed parts, like depressed skull fractures, over time, or to change the contour of a cartilaginous or fibrous part, like a cheekbone. Once the desired result is achieved, the magnet or magnets are removed.

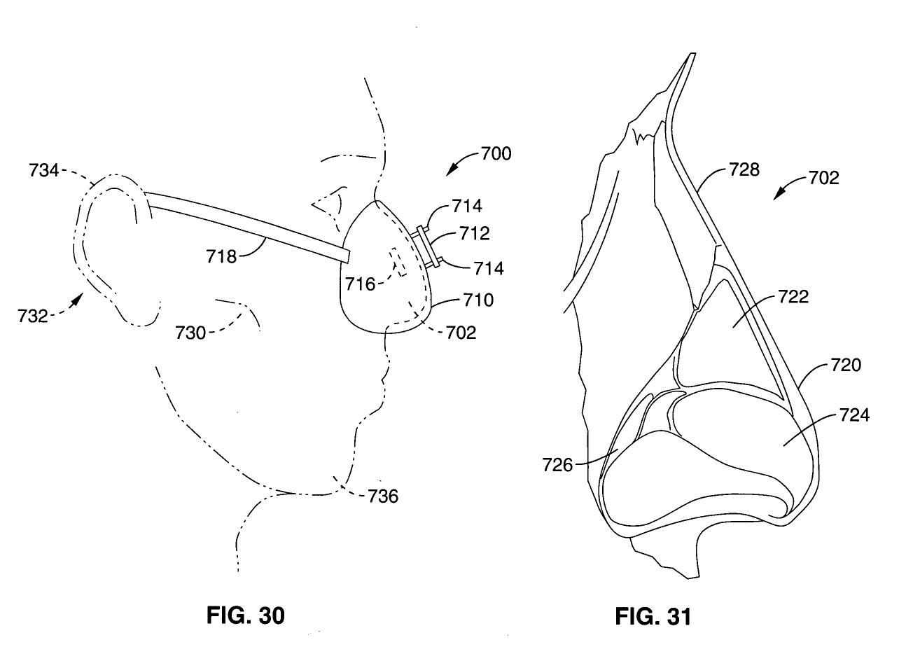

FIG. 30 illustrates a reformation device 700 for changing the physical appearance of a patient's nose. Like a rhinoplasty procedure, device 700 may be used to reduce or increase the size of the patient's nose 702, change the shape of the tip or the bridge of the nose, narrow the span of the nostrils, or change the angle between your nose and your upper lip. Device 700 may also be used to correct a birth defect or injury, or help relieve some breathing problems.

Reformation device 700 comprises a form 710 that is configured to mold the shape of the nose 702 via an applied pressure or tensile force over a period of time. The form 710 may be a preset shape based on the natural anatomy and desired shape, or could be custom molded via a desired shape chosen in advance by the patient.

The form 710 houses one or more external magnets 712, via adjustment members 714. Adjustment members 714, may be set screws, or the like adjustment means, mounted in a pattern that allows the magnet to be oriented angularly and away from the form 710 to adjust the force applied by the external magnet 714 or reposition the external magnet 714 after the patient's anatomy has shifted. The adjustment members 714 allow repeated adjustment of the magnet 712 and the applied load to the patient's anatomy.

Prior to placement of the form 710 for reformation, an implant having an internal magnetically-responsive member 716, such as a rear earth magnet or magnetically responsive metal, is installed under the patient's skin, or other anatomical feature of the patient. The implant may have a biocompatible coating or casing surrounding the internal magnet. Placement of the internal magnet 716 may vary depending on the desired physical reformation. For example, one or more internal magnets 716 may be positioned under the cartilaginous framework of the nose, e.g. the cartilage of the septum 720, the lateral cartilage 722, or the greater alar cartilage 724 or lesser alar cartilage 726 (see FIG. 31). These cartilaginous features are generally held together by a fibrous membrane, which may be stretched or manipulated by a small applied force over a period of time. Alternatively, the internal magnet 716 may be placed under the bony framework of the nose 724 to further affect reformation. The internal magnet 716 is ideally positioned and oriented such that a force will be applied in the direction of the desired manipulation, and a corresponding external magnet 712 will be positioned on the form 710 accordingly to affect such force.

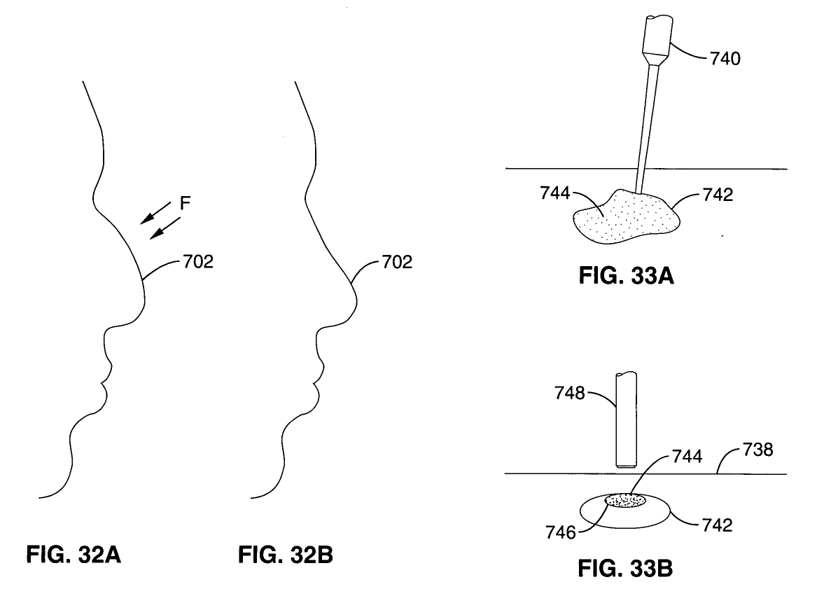

The internal magnet 716 and external magnet 714 may be positioned with opposite polarities facing each other to apply an attractive force, or a like polarities facing to form a repulsive force on the nose 702. For example, to perform a dorsal hump reduction as shown in FIG. 32 A and 32B, the magnets may be positioned to provide a repulsive force F and therefore depress the form 710 into the nose to minimize the nose profile. Alternatively, a depression in the nose may be excavated or pulled out into the form 710 via an attractive force between the internal magnet 716 and external magnet 714. Lateral forces may be applied as well by adjusting the placement of the magnets.

The internal magnet 716 may be implanted in a number of ways according to the desired implant location on the patient. For example, an incision may be performed inside the nostrils, or a small incision may be made across the columella (the vertical strip of tissue separating the nostrils) to gain access to the target implant location. Additional incisions or tissue manipulation may then be performed where appropriate to lodge the magnet in the appropriate locations. Sutures may be used to lock the magnet 716 in place once the target location is achieved.

Referring to FIGS. 33A-33B, a magnetically-charged internal implant may also be injected via a syringe 740 as a bio-compatible liquid gel 742, e.g. collagen, having a plurality of suspended magnetic particles 744. Once the solution is delivered to the target depth, e.g. subcutaneous, an external forming magnet 748 may be placed at or near the outside surface of the patient's skin 738 to concentrate the magnetic particles to form a magnetically-charged internal implant 746. The external forming magnet also serves to orient the magnetic particles so that their polarities are aligned.

Liquid gel 742 may comprise a bonding or thickening agent that solidifies the gel after a period of time. The external forming magnet 740 is retained in place until the get is set or solidified, and then removed, leaving the magnetic implant 746 incased in the gel 742. Although the gel may be sufficient in itself to isolate the magnetic particles from body tissues and fluids, the magnetic particles may further be coated with a biocompatible material, such as titanium or a titanium alloy.

These same principles may be applied to other regions of the body to reform a localized body feature. For example, custom forms 710 may be housing external magnets shaped to affect reformation of the auricular 734 of the ear 732, the chin 736, or cheekbone 730. Internal magnets 716 may be placed in corresponding locations and be configured such that they generate a repulsive force, e.g. for forming a depression, or an attractive force, e.g. for making a more pronounced chin or cheekbone normally accomplished through permanent implants.

One primary advantage of the reformation device 700 is that the form could be chosen in advance, and the patient could choose exactly how he or she would like the body part to look. Then the magnetic force is used to essentially mold the body part to the pre-made, pre-chosen form by applying small, comfortable loads to the target anatomy over a long period of time. For example, the form may be worn by the patient at night while the patient is sleeping, thus allowing the patient to undergo the reformation process with no public exposure.

The process is also considerably less invasive, involving minimal incision(s) (if necessary) for magnet placement. The traumatic tissue excavation and manipulation prevalent with current plastic surgery techniques would not be required. In addition, foreign implants, which are prone to failure, would also not be necessary. The negative stigma many potential patients associate with “going under the knife” would be minimized as the process of the present invention gradually “evolves” the patient anatomy over time, much similar physical anatomical changes achieved through weight training and the like.

6. Magnetic Jewelry

The above devices for magnetic alteration of anatomy and deformities may also be used to enable magnetic body art. By implanting magnetically responsive members, e.g. small magnets, in various parts of the body through a very small incision or injection through a needle, a number of ornaments, jewelry, tattoo-like art and even clothes can be hung reversibly and exchangably on the outside of the body. This process would be in lieu of body piercing because it would allow the same sort of ornamentation but without actually piercing the skin and the associated problems therewith.

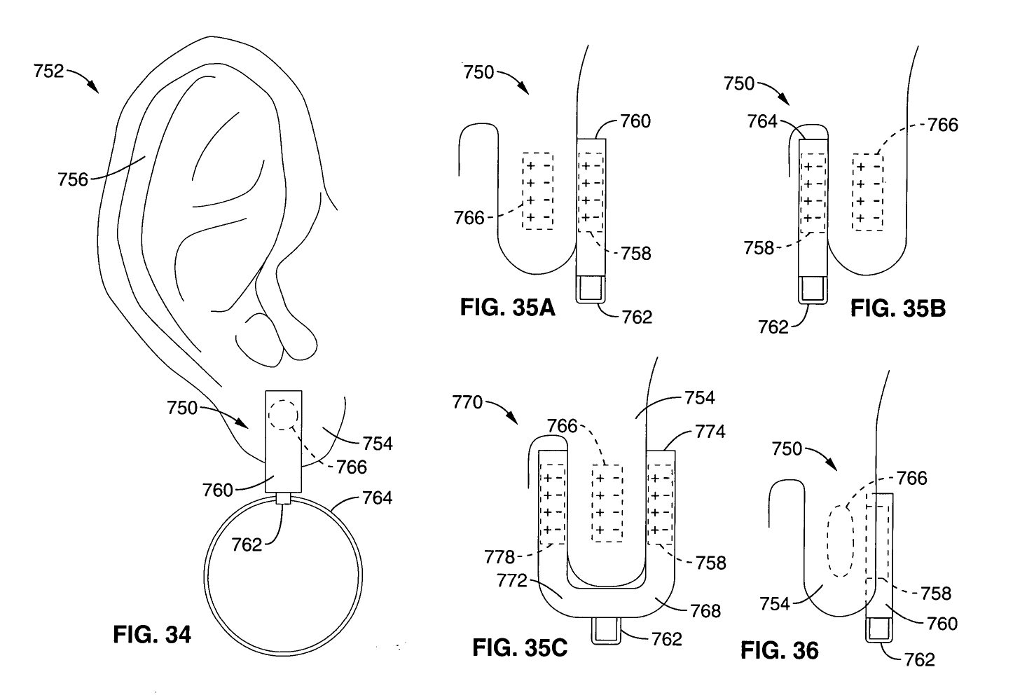

Referring now to FIGS. 34 and 35, a magnetic jewelry clasp 750 is illustrated in a fastened configuration on the earlobe 754 of a person's ear 752. A magnetically responsive magnetic implant 766 is first delivered to a region in or under the skin. This may be done via a small incision, or via a syringe and magnetic solution as detailed in FIGS. 33A and 33B.

The magnetic clasp 750 comprises a housing 760 that is configured to be placed adjacent on the outside surface of the earlobe 754. Housing 760 may comprise of a variety of materials, such as gold or silver, and preferably has a cutout or recess for housing an external magnet 766. Housing 760 also comprises a loop 762 for attachment of an ornamental piece 764. Loop 762 may also comprise a latch or clasp to allow exchange of ornamental piece 764, or simply provide a fastening point for another clasp.

As shown in FIG. 35A, the external magnet 758 and the internal magnet 766 are preferably positioned such that they face at opposite polarities to apply the attractive force holding the clasp 750 to the ear 752. In an alternative embodiment, a magnetic housing 764 may be configured to mount behind the earlobe 754, as shown in FIG. 35B.

In another configuration shown in FIG. 35C, magnetic clasp 770 may comprise a U-shaped housing 768 having a first external magnet 758 on an outer tine 774, and a second external magnet 778 on an inner tine 772. The first and second magnets are preferably configured such that each is facing in opposite polarity to internal magnet 766, effectively doubling the attractive force applied.

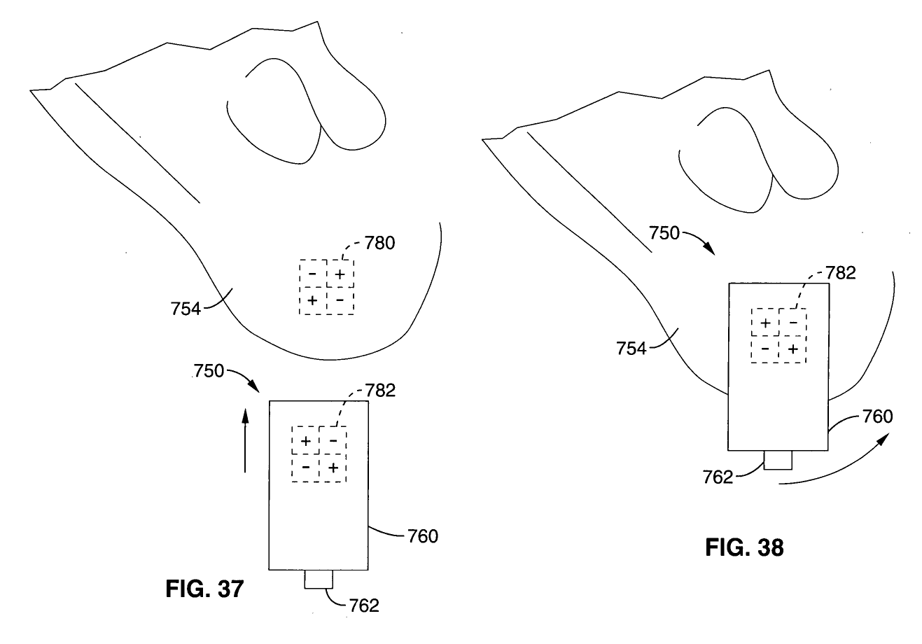

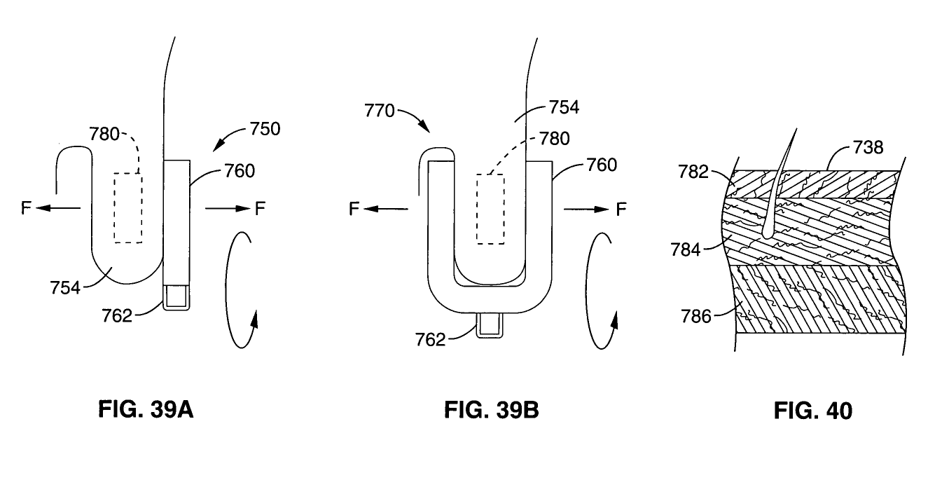

Referring now to FIG. 36, the housing 760 may compress the tissue between the internal magnet 766 and the external magnet 758. This may make the clasp difficult to remove. To ease removal, the internal 780 and external 782 magnets may be configured with opposite polarity quadrants, as shown in FIG. 37. To remove the magnetic clasp, the housing is rotated as shown in FIG. 38 such that the like polarities of the opposing magnets start to overlap, creating a repulsive force that either lifts the housing 760 off of the ear 752 as shown in FIG. 39A, or separates the tines 774 and 778 of clasp 770 away from the ear as shown in FIG. 39B.

The magnetic clasps describe above may be used on any part of the body for which the person wanted easily removable and exchangeable decoration. For example, a magnetic implant may be installed in the nose, lip, belly button, cheeks, legs, brows, or other desired body part. Larger magnets could also be used to hold larger items like designs that would mimic a tattoo or even clothing. The implantable magnets of the present invention would make it possible to allow clothing that gave the illusion of having no means of support like a strapless breast covering or small pieces of cloth used as swimwear for both males and females.

Internal magnets may also be used to fasten hair and toupees that would be exchangeable by virtue of magnetic fixation to a magnet implanted under the scalp. The above device may also be applied to a larger prosthetic body part, for instance, a prosthetic ear held on by magnetic attraction, or parts of soft tissue structures of a nose. It could also apply to decorative body parts, such as an abnormal or different ear or nose or even nipples or genitalia.

In other alternative embodiments, internal magnets may be implanted to magnetically fasten decorative parts of the body, such as teeth and fingernails. A thin magnet under the fingernail or attached to the top of the nail would allow new nail coverings of different shapes, sizes, and colors to be attached, unattached, and exchanged at will.

The internal implant may be implanted at varying depths depending on the application or location in the body. For example, FIG. 40 illustrates a cross-section of the human skin 738 and underlying tissue. To minimize distance between the external and internal magnets, the internal magnet may be implanted between the epidermis 782 and dermis 784 layers of the skin. For additional support from the skin or less external visibility, the implant may be placed between the subcutaneous 786 and dermis layers 784. For manipulation or reformation of other tissues such as that shown in FIG. 32, the magnet may be implanted below the subcutaneous fatty layer 786 or other tissue.

Although the description above contains many details, these should not be construed as limiting the scope of the invention but as merely providing illustrations of some of the presently preferred embodiments of this invention. For example, although the abovementioned embodiments primarily focus on pectus excavatum and scoliosis, the invention may be used on a variety of anatomical deformities. For example, pectus carinatum, scoliosis, club feet, cranial/facial anomalies or defects, skeletal dysplasias, cartilaginous deformities/dysplasias, and joint deformities/dysplasias may all be treated by the present invention. The invention may also be used to incrementally lengthen bone or apply bone compression to promote healing.

Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

1. An apparatus for incrementally manipulating an internal lumen of a patient, comprising:
a first magnetic member configured to be received at a first location on the lumen;
a first catheter adapted to deliver the first magnetic member to the first location, and
a second magnetic member adapted to be received at a second location of the lumen;
wherein the first and second magnetic members are configured to generate a magnetic force to incrementally lengthen at least a portion of the lumen.

2. An apparatus as recited in claim 1, wherein the first and second magnetic members comprise rare earth magnets.

3. An apparatus as recited in claim 1, wherein the first and second magnetic members are configured to bring two segments of the lumen together.

4. An apparatus as recited in claim 1, wherein the first and second magnetic members are configured to vary the force applied on the lumen.

5. An apparatus as recited in claim 4, wherein the first magnetic member is configured to vary its position with respect to the second magnetic member to vary the force applied.

6. An apparatus as recited in claim 5, wherein the first magnetic member is coupled to a linear actuation mechanism to vary its position with respect to the second magnetic member.

7. An apparatus as recited in claim 5, wherein the first magnetic member is encased in a biocompatible container; and
wherein the first magnet member is configured to be positioned at a plurality of locations inside the biocompatible container.

8. An apparatus as recited in claim 4, wherein the first magnetic member is responsive to an electric current; and
wherein said current may be varied to vary the force applied on the lumen.

9. An apparatus as recited in claim 4, wherein the first and second magnetic members are configured to cycle the force applied on the lumen.

10. An apparatus as recited in claim 4, further comprising a pressure sensor coupled to one of the first and second magnetic members, wherein the force applied to the lumen is adjusted according to a reading from the pressure sensor.

11. An apparatus as recited in claim 3, wherein the segments of the lumen comprise an upper esophageal atresia separated by a gap from a lower esophageal atresia; and
wherein the first and second magnetic members are configured to generate an attractive force to close the gap between the lower esophageal atresia and the upper esophageal atresia.

12. An apparatus as recited in claim 11, wherein the first and second magnetic members are configured to connect the lower esophageal atresia to the upper esophageal atresia.

13. An apparatus as recited in claim 11, wherein the first and second magnetic members are configured to anastomose portions of the lower esophageal atresia and the upper esophageal atresia.

14. An apparatus as recited in claim 11, wherein the first magnetic member is configured to be inserted trans-orally into the upper esophageal atresia via the first catheter;
and wherein the second magnetic member is configured to be inserted into the lower esophageal atresia through the patient's stomach via the second catheter.

15-23. (canceled)

24. An apparatus for incrementally manipulating a body member of a patient, comprising:
an implant adapted to be received on a location of the body member, the implant responsive to a magnetic field;
a form external to the patient; and
a magnetic member coupled to the form;
wherein the magnetic member is configured to generate a magnetic force between the implant and the form to incrementally manipulate the body member.

25. An apparatus as recited in claim 25, wherein the implant comprises a rare earth magnet.

26. An apparatus as recited in claim 26, wherein the magnetic member comprises a rare earth magnet.

27. An apparatus as recited in claim 25, wherein the implant and the magnetic member are configured to generate an attractive force between the implant and the form.

28. An apparatus as recited in claim 26, wherein the implant and the magnetic member are configured to generate a repulsive force between the implant and the platform.

29. An apparatus as recited in claim 25, wherein the magnetic force is configured to reposition the body member.

30. An apparatus as recited in claim 25, wherein the magnetic force is configured to deform the body member.

31. An apparatus as recited in claim 25:
wherein the body member comprises the patient's nose;
wherein the implant is adapted to be received at a location in the nose; and
wherein the form comprises a pre-determined shape adapted to be positioned exterior to the patient's nose to incrementally reform the shape of the nose to the pre-determined shape.

32. An apparatus as recited in claim 31, wherein the form is configured to reform a cartilaginous structure inside the nose.

33. An apparatus as recited in claim 31, wherein the form is configured to reform a bone structure inside the nose

34-43. (canceled)

44. A magnetic clasp for attaching an ornamental object to a body member, comprising:
a housing configured to be positioned against a skin surface at a location on the body member adjacent to a magnetically responsive implant positioned under the skin surface of the body member; and
a magnetic member coupled to the housing;
wherein the magnetic member is configured to generate an attractive force between the implant and the housing to retain the housing at the location.

45. An apparatus as recited in claim 44, wherein the housing comprises the ornamental object.

46. An apparatus as recited in claim 44, wherein the housing is configured to retain the ornamental object near the location.

47. An apparatus as recited in claim 46, wherein the housing is configured to detachably secure the ornamental object near the location.

48. An apparatus as recited in claim 44, wherein the body member comprises an earlobe, and wherein the ornamental object comprises an earring.

49-56. (canceled)
